(19) United States
(12) Patent Application Publication
(10) Pub. No.: US 2026/0116939 A1
(43) Pub. Date: Apr. 30, 2026

(54) CELL SIGNALING COMPLEXES AND USES THEREOF

(71) Applicant: Nanotein Technologies, Inc., Berkeley, CA (US)

(72) Inventors: Curtis Daniel HODGE, Pinole, CA (US); Zachary Ibrahim IMAM, Concord, CA (US); Luke Garland HAINES, Pleasant Hill, CA (US)

(73) Assignee: Nanotein Technologies, Inc., Berkeley, CA (US)

(21) Appl. No.: 19/010,892

(22) Filed: Jan. 6, 2025

Related U.S. Application Data
(62) Division of application No. 18/731,180, filed on May 31, 2024, now Pat. No. 12,227,552.
(60) Provisional application No. 63/511,968, filed on Jul. 5, 2023.

(51) Int. Cl. C07K 14/54; C07K 14/55; C12N 5/0783

(52) U.S. Cl. CPC C07K 14/5443; C07K 14/5418; C07K 14/55; C12N 5/0636; C07K 2319/02; C07K 2319/30; C12N 2501/2302; C12N 2501/2307; C12N 2501/2315

(57) ABSTRACT

Disclosed herein are cell signaling complexes such as chimeric cytokine complexes and uses thereof. In some embodiments, a chimeric cytokine complex comprises a protein cage polypeptide, a plurality of engineered Fc antibody domains bound to the protein cage polypeptide, and one or more cytokines linked to each of the plurality of engineered Fc antibody domains. Also disclosed are Fc-cytokine complexes and uses thereof. In some embodiments, an Fc-cytokine complex comprises an engineered Fc antibody domain and one or more cytokines linked to the engineered Fc antibody domain.

### CROSS-REFERENCE TO RELATED APPLICATIONS

This application is a divisional of U.S. patent application Ser. No. 18/731,180 filed on May 31, 2024, which claims the benefit of U.S. Patent Application No. 63/511,968 filed on Jul. 5, 2023, the contents of which are incorporated herein by reference in their entireties.

### REFERENCE TO AN ELECTRONIC SEQUENCE LISTING

This application contains a Sequence Listing which has been submitted via Patent Center and is hereby incorporated by reference in its entirety. Said .xml copy, created on May 15, 2024 is named 05_NNTNNZ00101_sequence_listing.xml and is 35,143 bytes in size.

### TECHNICAL FIELD

This disclosure relates generally to the field of cell signaling complexes and, more specifically, to cell signaling complexes such as chimeric cytokine complexes and uses thereof.

### BACKGROUND

A commonly used mechanism for cellular pathway activation from a cell surface is receptor oligomerization or receptor clustering. For example, receptor oligomerization/clustering is demonstrated through co-stimulatory receptor binding by ligands and agonistic antibodies [1] and by the binding of cell signaling molecules such as cytokines to certain cell receptors [2]. These receptors often include an extracellular domain on the surface of a cell, a transmembrane domain that passes through the cellular membrane, and an intracellular domain on the inside of the cell. In these receptor clustering paradigms, the extracellular domain of the receptors are engaged by cell signaling molecules, which can cause both conformational changes that translate through the membrane to contribute to activation/signaling, and/or oligomerization of multiple receptors that bring together intracellular signaling complexes that drive activation/signaling.

As previously discussed, cytokines are a well-known class of cell signaling proteins. The well-established canonical mechanism for cytokines is the dimerization of extracellular cytokine receptors, often through engagement of two JAK (Janus kinase)-associated receptor subunits in a heterodimeric and sometimes homodimeric fashion [2, 3]. JAK dimerization leads to phosphorylation of STAT (signal transducer and activator of transcription) transcription factors to drive cellular activation through gene regulation [4].

Cytokines can further be classified into interleukins, interferons, chemokines, lymphokines, colony-stimulation factors (CSFs), and tumor necrosis factors (TGFs). One important family of cytokines is the common γ-chain (γc) family of cytokines that play an important role in T cell expansion and survival. Some of the members of the ye family of cytokines include interleukin-2 (IL-2), IL-7, and IL-15.

The functional IL-2-engaged IL-2 receptor signaling complex can consist of sequential binding/complexing of IL-2Rα, IL-2Rβ, and γc subunits as a high-affinity heterotrimer (K_{d}˜10⁻¹¹M) or an ˜100-fold intermediate affinity dimeric complex of IL-2Rβ, and γc only (K_{d}˜10⁻⁹M) [2, 5]. IL-2 has a low affinity to the IL-2Rα subunit (K_{d}˜10⁻⁸M), but this binding does not induce signaling. Naïve T cells are thought to start out with low levels of IL-2Rα, but upon T cell receptor (TCR) activation, IL-2Rα expression is upregulated. IL-2Rβ, and γc are constitutively expressed on lymphohematopoietic cells, which include low-density expression on naïve T cells [5, 6]. In the activated T cell scenario, IL-2 will engage with the upregulated IL-2Rα subunit and the lowly expressed, intermediate affinity IL-2Rβ, and γc heterodimer, leading to low activation in the later or inefficient recruitment of all receptor subunits to assemble the high-affinity signaling complex. Thus, approaches to increase IL-2 receptor signaling could take the form of: (i) increasing the affinity of IL-2 to the signaling-capable IL-2Rβ/γc heterodimer to effectively increase the signaling stability, (ii) increasing the recruitment and assembly of all three IL-2 receptor subunits to form the high-affinity signaling complex, or (iii) increasing the number of productive intermediate and high-affinity signaling complexes on the cell surface.

While there have been successful cytokine engineering efforts aimed at modulating receptor signaling by focusing on modulating IL-2's receptor binding affinity [2, 6], these efforts have focused on direct amino acid changes to the cytokine sequences to disrupt or enhance binding affinities to specific receptor subunits. Such efforts often require labor-intensive and time-intensive techniques such as directed evolution, X-ray crystallography, molecular dynamics simulations, and iterative cytokine-receptor interface engineering [2, 6], while only really taking advantage of the affinity approach. An example of these sequence-based engineering efforts is the development of interleukin-2 (IL-2) variants dubbed “superkines” or super-2s [6].

Therefore, a solution is needed that takes advantage of the various approaches for inducing cytokine receptor signaling described above with respect to increased IL-2 receptor signaling without having to make sequence-based changes to the cytokines themselves. Moreover, such a solution should increase the affinity of cytokines to their signaling receptors while also increasing the overall number of cytokine-receptor signaling complexes per T cell. Furthermore, such a solution should result in increased activation and expansion of human immune cells.

### SUMMARY

Disclosed are improved cell signaling complexes capable of facilitating the activation and expansion of immune cells. More specifically, disclosed are cell signaling complexes capable of facilitating the activation and expansion of immune cells (e.g., human donor peripheral blood T cells, human peripheral blood NK cells, etc.).

In some embodiments, a chimeric cytokine complex comprises a protein cage polypeptide, a plurality of engineered Fc antibody domains bound to the protein cage polypeptide, and one or more cytokines linked to each of the plurality of engineered Fc antibody domains.

Also disclosed is a method of activating and expanding immune cells. The method can comprise adding a chimeric cytokine complex to a population of immune cells. The chimeric cytokine complex can comprise a protein cage polypeptide, a plurality of engineered Fc antibody domains bound to the protein cage polypeptide, and one or more cytokines linked to each of the plurality of Fc antibody domains.

In some embodiments, the population of immune cells can be activated and expanded in vitro. In various embodiments, the population of immune cells can be human donor peripheral blood immune cells.

In various embodiments, the population of immune cells can be live T cells.

In various embodiments, the population of immune cells can be natural killer (NK) cells.

In some embodiments, the method can further comprise activating the population of T cells with an anti-CD3 and anti-CD28 T-cell activation reagent prior to adding the chimeric cytokine complex.

In various embodiments, the cytokines can be interleukins. The interleukins can be at least one of interleukin-2s (IL-2s), IL-7s, and IL-15s.

In various embodiments, the plurality of engineered Fc antibody domains can comprise between six and twelve engineered Fc antibody domains bound to the protein cage polypeptide. In some embodiments, between 12 and 24 cytokines can be linked to the plurality of engineered Fc antibody domains in total.

In various embodiments, each of the one or more cytokines can be linked to one of the engineered Fc antibody domains via an engineered metalloprotease-resistant linker sequence. In some embodiments, the engineered metalloprotease-resistant linker sequence can be between 7 to 13 amino acid residues in length.

In various embodiments, at least one of the engineered Fc antibody domains can be C-terminally linked to an N-terminus of at least one of the cytokines.

In some embodiments, one of the cytokines can be interleukin-2 (IL-2) and at least one of the engineered Fc antibody domains can be linked to the IL-2 via an engineered metalloprotease-resistant linker sequence comprising the amino acid sequence SLSPGKAPTS (SEQ ID NO:20).

In some embodiments, one of the cytokines is interleukin-7 (IL-7) and at least one of the engineered Fc antibody domains can be linked to the IL-7 via an engineered metalloprotease-resistant linker sequence comprising the amino acid sequence SLSPGKDCDIEGK (SEQ ID NO:21).

In some embodiments, one of the cytokines can be interleukin-15 (IL-15) and at least one of the engineered Fc antibody domains can be linked to the IL-15 via an engineered metalloprotease-resistant linker sequence comprising the amino acid sequence SLSPGKN (SEQ ID NO:22).

In some embodiments, at least one of the engineered Fc antibody domains can be N-terminally linked to a C-terminus of at least one of the cytokines.

In some embodiments, one of the cytokines can be interleukin-2 (IL-2) and at least one of the engineered Fc antibody domains can be linked to the IL-2 via an engineered metalloprotease-resistant linker sequence comprising the amino acid sequence TPKSCDKTHT (SEQ ID NO:23).

In some embodiments, one of the cytokines can be interleukin-7 (IL-7) and at least one of the engineered Fc antibody domains can be linked to the IL-7 via an engineered metalloprotease-resistant linker sequence comprising the amino acid sequence HPKSCDKTHT (SEQ ID NO:24).

In some embodiments, one of the cytokines can be interleukin-15 (IL-15) and at least one of the engineered Fc antibody domains can be linked to the IL-15 via an engineered metalloprotease-resistant linker sequence comprising the amino acid sequence TSPKSCDKTHT (SEQ ID NO:25).

In various embodiments, the engineered Fc antibody domains can be engineered human Fc antibody domains.

In some embodiments, the engineered human Fc antibody domains can be engineered human IgG1 Fc antibody domains.

In various embodiments, one of the engineered human IgG1 Fc antibody domains can comprise an amino acid sequence with at least about 70%, 75%, 80%, 85%, 90%, 95%, 99%, or 100% amino acid identity to the amino acid sequence set forth in SEQ ID NO:1.

In some embodiments, the engineered human IgG1 Fc antibody domain can comprise at least one of the following point mutations relative to SEQ ID NO:1 to decrease affinity for certain Fcγ receptors and functionally reduce antibody-dependent cellular cytotoxicity (ADCC): P75L, R76W, Y80K, Y80P, Y80R, Y80G, and Y80A.

In some embodiments, the engineered human IgG1 Fc antibody domain can comprise the following point mutation relative to SEQ ID NO:1 to increase affinity for certain Fcγ receptors and functionally increase antibody-dependent cellular cytotoxicity (ADCC): Y80W.

In some embodiments, the engineered human IgG1 Fc antibody domain can comprise at least one of the following point mutations relative to SEQ ID NO:1 to decrease affinity for certain Fcγ receptors and have a neutral effect on other Fcγ receptors: S23A, E53A, E77A, Y80F, V87A, A111G, K122A, and D160A.

In some embodiments, the engineered human IgG1 Fc antibody domain can comprise at least one of the following point mutations relative to SEQ ID NO:1 to increase affinity for certain Fcγ receptors and have a neutral effect on other Fcγ receptors: E117, K118A, and A123T.

In some embodiments, the engineered human IgG1 Fc antibody domain can comprise at least one of the following point mutations relative to SEQ ID NO:1 to increase affinity for certain Fcγ receptors and decrease affinity for certain other Fcγ receptors: H52A, R85A, and K106A.

In some embodiments, the engineered human IgG1 Fc antibody domain can comprise at least one of the following point mutations relative to SEQ ID NO:1 to decrease affinity for certain Fcγ receptors: D54A, Q79A, and A111S.

In some embodiments, the engineered human IgG1 Fc antibody domain can comprise at least one of the following point mutations relative to SEQ ID NO:1 to increase affinity for certain Fcγ receptors: T40A and K74A.

In some embodiments, the Fc antibody domains can be engineered rabbit Fc antibody domains.

In various embodiments, the chimeric cytokine complex can further comprise a signal peptide linked to an N-terminus of at least one of the engineered Fc antibody domains or at least one of the cytokines.

In some embodiments, the signal peptide can be a murine Ig heavy signal peptide used for expression in Chinese hamster ovary (CHO) cells. In some embodiments, the signal peptide can comprise the amino acid sequence MGWSCIILFLVATATGVHS (SEQ ID NO:26).

In various embodiments, the protein cage polypeptide can comprise a polypeptide comprising an amino acid sequence with at least about 70%, 75%, 80%, 85%, 90%, 95%, 99%, or 100% amino acid identity to the amino acid sequence set forth in any one of SEQ ID NOS: 8-15.

In some embodiments, the amino acid sequence making up the polypeptide of the protein cage polypeptide can comprise at least the following point mutation relative to the amino acid sequence set forth in any one of SEQ ID NOS: 8-15: Y294A.

In some embodiments, the protein cage polypeptide can comprise a polypeptide comprising a binding site for one of the engineered Fc antibody domains. The binding site can comprise the amino acid sequence RWGSGADCAWHLGELVWCTAGSGWE (SEQ ID NO:16).

In some embodiments, the protein cage polypeptide can comprise a polypeptide comprising a binding site for one of the engineered Fc antibody domains. The binding site can comprise the amino acid sequence GGRWGADCAWHLGELVWCTAGWEGG (SEQ ID NO:17).

In some embodiments, the protein cage polypeptide can comprise a polypeptide comprising a binding site for one of the engineered Fc antibody domains. The binding site can comprise the amino acid sequence GADCAWHLGELVWCTAG (SEQ ID NO:18).

In some embodiments, the protein cage polypeptide can comprise a polypeptide comprising a binding site for one of the engineered Fc antibody domains. The binding site can comprise the amino acid sequence RWGSGCDCAWHLGELVWCTCGSGWE (SEQ ID NO:19).

In some embodiments, the protein cage polypeptide can self-assemble into a tetrahedral pyramid structure.

In some embodiments, an Fc-cytokine complex is disclosed comprising an engineered Fc antibody domain and one or more cytokines linked to the engineered Fc antibody domain.

Also disclosed is a method of activating and expanding immune cells using the Fc-cytokine complex. The method can comprise adding an Fc-cytokine complex to a population of immune cells. The Fc-cytokine complex can comprise an Fc antibody domain and one or more cytokines linked to the Fc antibody domain.

In some embodiments, the population of immune cells can be activated and expanded in vitro. In various embodiments, the population of immune cells can be human donor peripheral blood immune cells. In various embodiments, the population of immune cells can be live T cells.

In some embodiments, the population of immune cells can be natural killer (NK) cells.

In some embodiments, the method can further comprise activating the population of T cells with an anti-CD3 and anti-CD28 T-cell activation reagent prior to adding the Fc-cytokine complex.

In various embodiments, the cytokines can be interleukins.

In some embodiments, the interleukins can be at least one of interleukin-2s (IL-2s), IL-7s, and IL-15s.

In various embodiments, each of the one or more cytokines can be linked to the engineered Fc antibody domain via an engineered metalloprotease-resistant linker sequence. In some embodiments, the engineered metalloprotease-resistant linker sequence can be between 7 to 13 amino acid residues in length.

In various embodiments, the engineered Fc antibody domain can be C-terminally linked to an N-terminus of at least one of the cytokines.

In some embodiments, one of the cytokines can be interleukin-2 (IL-2) and the engineered Fc antibody domain can be linked to the IL-2 via an engineered metalloprotease-resistant linker sequence comprising the amino acid sequence SLSPGKAPTS (SEQ ID NO:20).

In some embodiments, one of the cytokines can be interleukin-7 (IL-7) and the engineered Fc antibody domain can be linked to the IL-7 via an engineered metalloprotease-resistant linker sequence comprising the amino acid sequence SLSPGKDCDIEGK (SEQ ID NO:21).

In some embodiments, one of the cytokines can be interleukin-15 (IL-15) and the engineered Fc antibody domain can be linked to the IL-15 via an engineered metalloprotease-resistant linker sequence comprising the amino acid sequence SLSPGKN (SEQ ID NO:22).

In various embodiments, the engineered Fc antibody domain can be N-terminally linked to a C-terminus of at least one of the cytokines.

In some embodiments, one of the cytokines can be interleukin-2 (IL-2) and the engineered Fc antibody domain can be linked to the IL-2 via an engineered metalloprotease-resistant linker sequence comprising the amino acid sequence TPKSCDKTHT (SEQ ID NO:23).

In some embodiments, one of the cytokines can be interleukin-7 (IL-7) and the engineered Fc antibody domain can be linked to the IL-7 via an engineered metalloprotease-resistant linker sequence comprising the amino acid sequence HPKSCDKTHT (SEQ ID NO:24).

In some embodiments, one of the cytokines can be interleukin-15 (IL-15) and the engineered Fc antibody domain can be linked to the IL-15 via an engineered metalloprotease-resistant linker sequence comprising the amino acid sequence TSPKSCDKTHT (SEQ ID NO:25).

In various embodiments, the engineered Fc antibody domain can be an engineered human Fc antibody domain. In some embodiments, the engineered human Fc antibody domain can be an engineered human IgG1 Fc antibody domain.

In some embodiments, the engineered human IgG1 Fc antibody domain can comprise an amino acid sequence with at least about 70%, 75%, 80%, 85%, 90%, 95%, 99%, or 100% amino acid identity to the amino acid sequence set forth in SEQ ID NO:1.

In some embodiments, the engineered human IgG1 Fc antibody domain can comprise at least one of the following point mutations relative to SEQ ID NO:1 to decrease affinity for certain Fcγ receptors and functionally reduce antibody-dependent cellular cytotoxicity (ADCC): P75L, R76W, Y80K, Y80P, Y80R, Y80G, and Y80A.

In some embodiments, the engineered human IgG1 Fc antibody domain can comprise the following point mutation relative to SEQ ID NO:1 to increase affinity for certain Fcγ receptors and functionally increase antibody-dependent cellular cytotoxicity (ADCC): Y80W.

In some embodiments, the engineered human IgG1 Fc antibody domain can comprise at least one of the following point mutations relative to SEQ ID NO:1 to decrease affinity for certain Fcγ receptors and have a neutral effect on other Fcγ receptors: S23A, E53A, E77A, Y80F, V87A, A111G, K122A, and D160A.

In some embodiments, the engineered human IgG1 Fc antibody domain can comprise at least one of the following point mutations relative to SEQ ID NO:1 to increase affinity for certain Fcγ receptors and have a neutral effect on other Fcγ receptors: E117, K118A, and A123T.

In some embodiments, the engineered human IgG1 Fc antibody domain can comprise at least one of the following point mutations relative to SEQ ID NO:1 to increase affinity for certain Fcγ receptors and decrease affinity for certain other Fcγ receptors: H52A, R85A, and K106A.

In some embodiments, the engineered human IgG1 Fc antibody domain can comprise at least one of the following point mutations relative to SEQ ID NO:1 to decrease affinity for certain Fcγ receptors: D54A, Q79A, and A111S.

In some embodiments, the engineered human IgG1 Fc antibody domain can comprise at least one of the following point mutations relative to SEQ ID NO:1 to increase affinity for certain Fcγ receptors: T40A and K74A.

In some embodiments, the Fc antibody domain can be an engineered rabbit Fc antibody domain.

In various embodiments, the Fc-cytokine complex can further comprise a signal peptide linked to an N-terminus of the engineered Fc antibody domain or at least one of the cytokines. In some embodiments, the signal peptide can be a murine Ig heavy signal peptide used for expression in Chinese hamster ovary (CHO) cells. In some embodiments, the signal peptide can comprise the amino acid sequence MGWSCIILFLVATATGVHS (SEQ ID NO:26).

### BRIEF DESCRIPTION OF THE DRAWINGS

FIGS. 1A and 1B illustrate predicted structures that represent one embodiment of an engineered Fc antibody domain linked to a plurality of cytokines.

FIG. 1C illustrates a predicted structure of one embodiment of an engineered self-assembling protein cage polypeptide.

FIG. 1D illustrates a predicted structure of one embodiment of a cytokine (e.g., IL-2) and its receptor signaling complex.

FIG. 1E illustrates a predicted structure of one embodiment of a chimeric cytokine complex (CCC) comprising an Fc-cytokine bound to an engineered self-assembling protein cage polypeptide and a receptor signaling complex bound to the cytokine of the CCC.

FIG. 2 illustrates that the engineered Fc antibody domains can be C-terminally linked or N-terminally linked to the cytokines to achieve their augmented function of activating and expanding T cells.

FIG. 3 illustrates example linker sequences designed to link the Fc and cytokine components of various CCCs.

FIG. 4 illustrates an example of an engineered Fc component of the CCC stabilized by an Fcγ receptor (FcγR) of a nearby cell.

FIG. 5 illustrates that the Fc component of the CCC can be engineered through mutation of key sites in its FcγR-binding portion.

FIG. 6 is a graph illustrating the results of an ELISA specific for detecting IFN-γ produced by T cells. A phosphate buffered saline (PBS) solution was used as the control.

FIG. 7 is a graph illustrating the population of live T cells as determined using flow cytometry on day 7. The T cells were activated and expanded using CCCs, Fc-IL-2s, and IL-2s alone. A phosphate buffered saline (PBS) solution was used as the control.

FIG. 8 is a graph illustrating the population of live T cells as determined using flow cytometry on day 7. The T cells were activated and expanded using a combination of CCCs and cytokines and cytokines alone. A phosphate buffered saline (PBS) solution was used as the control.

FIGS. 9A-9C are graphs illustrating the CD4+ T cell count, the CD8+ T cell count, and the total live T cells at day 7, respectively, of T cells activated and expanded by CCCs and cytokines alone. A phosphate buffered saline (PBS) solution was used as the control.

FIG. 10 is a graph showing that upon stimulation with an anti-CD3 and anti-CD28 T-cell activation reagent, peripheral blood donor T cells upregulated the expression of cytokines and chemokines that increase the expression of certain matrix metalloproteases (MMPs) by T cells.

FIGS. 11A-11D illustrate that NK cells on media supplemented with CCCs proliferated much more than NK cells on media supplemented with standard soluble cytokines alone or Fc-cytokines after three days of coincubation.

### DETAILED DESCRIPTION

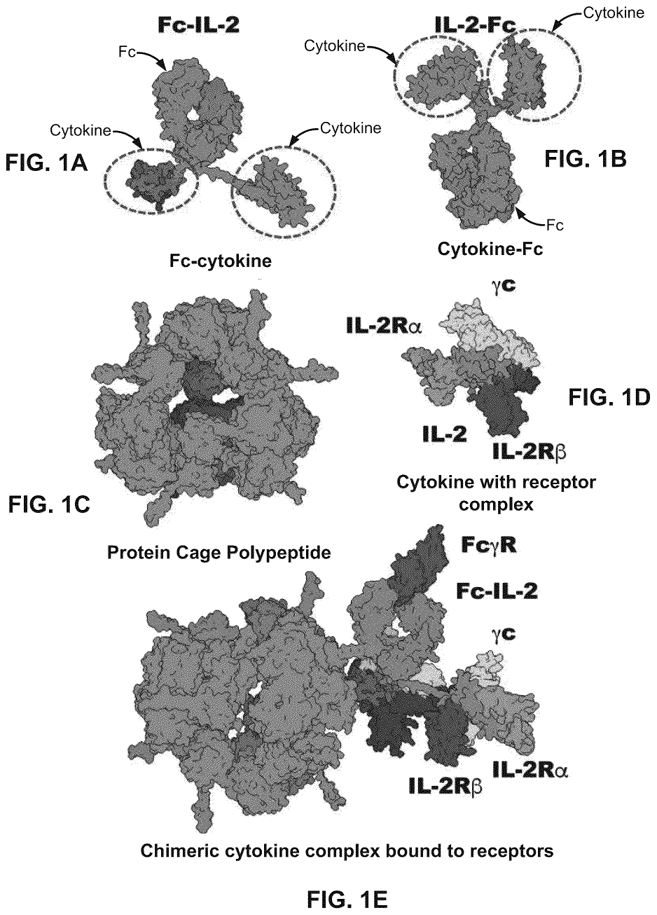

FIGS. 1A and 1B illustrate predicted structures that represent one embodiment of an engineered Fc antibody domain linked to a plurality of cytokines. For example, FIG. 1A illustrates a predicted structure of an engineered Fc antibody domain (e.g., an engineered IgG1 Fc heavy chain) C-terminally linked to two IL-2 cytokines. Also, for example, FIG. 1B illustrates a predicted structure of an engineered Fc antibody domain (e.g., an engineered IgG1 Fc heavy chain) N-terminally linked to two IL-2 cytokines.

In some embodiments, the engineered Fc antibody domain can be an engineered human Fc antibody domain. For example, the Fc antibody domain can be an engineered human IgG1 Fc antibody domain.

In some embodiments, the engineered human IgG1 Fc antibody domain can comprise an amino acid sequence with at least about 70%, 75%, 80%, 85%, 90%, 95%, 99%, or 100% amino acid identity to the amino acid sequence set forth in SEQ ID NO:1 (see Table 1).

In other embodiments, the Fc antibody domain can be an engineered rabbit Fc antibody domain. For example, the Fc antibody domain can be an engineered rabbit IgG Fc antibody domain.

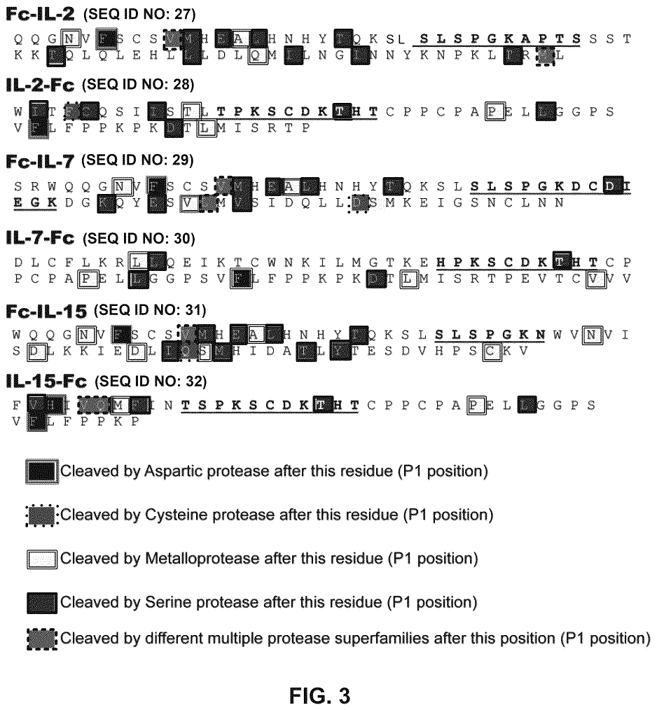

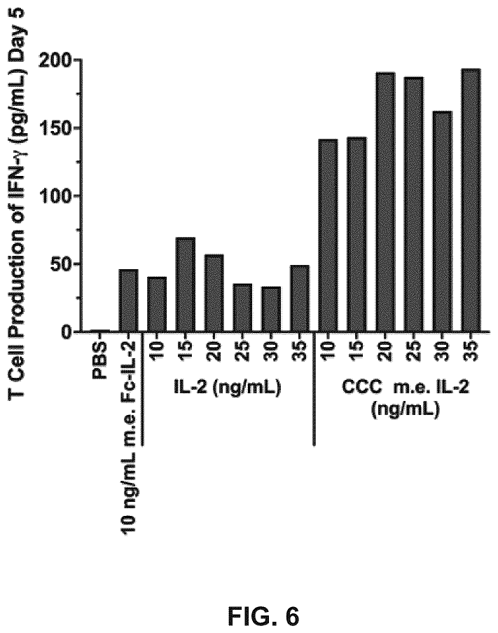

Although FIGS. 1A and 1B illustrate the cytokines as IL-2s, it is contemplated by this disclosure that other interleukins such as IL-7s and IL-15s (see, e.g., FIGS. 3 and 6) can also act as the cytokines. Moreover, it is contemplated by this disclosure that other receptor-engaging molecules (engineered or natural) can be used as the cell signaling component of the CCCs.

FIG. 1C illustrates a predicted structure of an engineered self-assembling protein cage polypeptide. As shown in FIG. 1C, the protein cage polypeptide can self-assemble into a tetrahedral pyramid structure. The protein cage polypeptide can also self-assemble into a compact asymmetrical multimeric structure or a cage-cage multimer (including dimers).

The protein cage polypeptide can be any of the protein cage polypeptides or scaffolding proteins discussed in U.S. Patent Publication No. 2022/0196655, the content of which is incorporated herein by reference in its entirety.

The engineered self-assembling protein cage polypeptide can serve as a carrier or scaffold for a plurality of engineered Fc antibody domains, each linked to one or more cytokines. When the plurality of engineered Fc antibody domains (each linked to one or more cytokines) are bound to the protein cage polypeptide, such a structure is referred to herein as a chimeric cytokine complex (CCC).

FIG. 1D illustrates an example of a cytokine (e.g., IL-2) and its receptor signaling complex. In some embodiments, the receptor signaling complex can comprise IL-2Rα, IL-2Rβ, and γc. In other embodiments, the receptor complex can be the dimeric complex of IL-2Rβ, and γc only.

FIG. 1E illustrates a predicted structure of an example CCC comprising an Fc-cytokine bound to an engineered self-assembling protein cage polypeptide and a receptor signaling complex bound to the cytokine of the CCC (e.g., IL-2). The protein cage polypeptide can comprise a plurality of potential binding sites for the engineered Fc antibody domains. For example, the protein cage polypeptide can comprise up to 12 binding sites for Fc antibody domains.

Although FIG. 1E illustrates only one Fc-cytokine chimera (e.g., Fc-IL-2) bound to the protein cage polypeptide, it is contemplated by this disclosure that between six and twelve engineered Fc antibody domains can be bound to one protein cage polypeptide.

Since each engineered Fc antibody domain can comprise up to two cytokines, each CCC can comprise between 12 and 24 cytokines.

TABLE 1
Engineered Fc sequence and engineered Fc-cytokine sequences
designed and experimentally tested to-date.

| SEQ ID NO: | NAME | SEQUENCE |
|---|---|---|
| 1 | Engineered | PKSCDKTHTCPPCPAPELLGGPSVFLFPPKPKDTLMISRTPE |
| | Human | VTCVVVDVSHEDPEVKFNWYVDGVEVHNAKTKPREEQY |
| | IgG1 Fc | NSTYRVVSVLTVLHQDWLNGKEYKCKVSNKALPAPIEKT |
| | | ISKAKGQPREPQVYTLPPSRDELTKNQVSLTCLVKGFYPS |
| | | DIAVEWESNGQPENNYKTTPPVLDSDGSFFLYSKLTVDKS |
| | | RWQQGNVFSCSVMHEALHNHYTQKSLSLSPGK |
| 2 | Engineered | MGWSCIILFLVATATGVHSPKSCDKTHTCPPCPAPELLGGP |
| | Fc-IL-2 | SVFLFPPKPKDTLMISRTPEVTCVVVDVSHEDPEVKENWY |
| | | VDGVEVHNAKTKPREEQYNSTYRVVSVLTVLHQDWLNG |
| | | KEYKCKVSNKALPAPIEKTISKAKGQPREPQVYTLPPSRDE |
| | | LTKNQVSLTCLVKGFYPSDIAVEWESNGQPENNYKTTPPV |
| | | LDSDGSFFLYSKLTVDKSRWQQGNVFSCSVMHEALHNHY |
| | | TQKSLSLSPGKAPTSSSTKKTQLQLEHLLLDLQMILNGINN |
| | | YKNPKLTRMLTFKFYMPKKATELKHLQCLEEELKPLEEV |
| | | LNLAQSKNFHLRPRDLISNINVIVLELKGSETTFMCEYADE |
| | | TATIVEFLNRWITFCQSIISTLT |
| 3 | Engineered | MGWSCIILFLVATATGVHSAPTSSSTKKTQLQLEHLLLDL |
| | IL-2-Fc | QMILNGINNYKNPKLTRMLTFKFYMPKKATELKHLQCLE |
| | | EELKPLEEVLNLAQSKNFHLRPRDLISNINVIVLELKGSETT |
| | | FMCEYADETATIVEFLNRWITFCQSIISTLTPKSCDKTHTCP |
| | | PCPAPELLGGPSVFLFPPKPKDTLMISRTPEVTCVVVDVSH |
| | | EDPEVKFNWYVDGVEVHNAKTKPREEQYNSTYRVVSVL |
| | | TVLHQDWLNGKEYKCKVSNKALPAPIEKTISKAKGQPREP |
| | | QVYTLPPSRDELTKNQVSLTCLVKGFYPSDIAVEWESNGQ |
| | | PENNYKTTPPVLDSDGSFFLYSKLTVDKSRWQQGNVFSCS |
| | | VMHEALHNHYTQKSLSLSPGK |
| 4 | Engineered | MGWSCIILFLVATATGVHSPKSCDKTHTCPPCPAPELLGGP |
| | Fc-IL-7 | SVFLFPPKPKDTLMISRTPEVTCVVVDVSHEDPEVKFNWY |
| | | VDGVEVHNAKTKPREEQYNSTYRVVSVLTVLHQDWLNG |
| | | KEYKCKVSNKALPAPIEKTISKAKGQPREPQVYTLPPSRDE |
| | | LTKNQVSLTCLVKGFYPSDIAVEWESNGQPENNYKTTPPV |
| | | LDSDGSFFLYSKLTVDKSRWQQGNVFSCSVMHEALHNHY |
| | | TQKSLSLSPGKDCDIEGKDGKQYESVLMVSIDQLLDSMKE |
| | | IGSNCLNNEFNFFKRHICDANKEGMFLFRAARKLRQFLKM |
| | | NSTGDFDLHLLKVSEGTTILLNCTGQVKGRKPAALGEAQP |
| | | TKSLEENKSLKEQKKLNDLCFLKRLLQEIKTCWNKILMGT |
| | | KEH |
| 5 | Engineered | MGWSCIILFLVATATGVHSDCDIEGKDGKQYESVLMVSID |
| | IL-7-Fc | QLLDSMKEIGSNCLNNEFNFFKRHICDANKEGMFLFRAAR |
| | | KLRQFLKMNSTGDFDLHLLKVSEGTTILLNCTGQVKGRKP |
| | | AALGEAQPTKSLEENKSLKEQKKLNDLCFLKRLLQEIKTC |
| | | WNKILMGTKEHPKSCDKTHTCPPCPAPELLGGPSVFLFPP |
| | | KPKDTLMISRTPEVTCVVVDVSHEDPEVKFNWYVDGVEV |
| | | HNAKTKPREEQYNSTYRVVSVLTVLHQDWLNGKEYKCK |
| | | VSNKALPAPIEKTISKAKGQPREPQVYTLPPSRDELTKNQV |
| | | SLTCLVKGFYPSDIAVEWESNGQPENNYKTTPPVLDSDGS |
| | | FFLYSKLTVDKSRWQQGNVFSCSVMHEALHNHYTQKSLS |
| | | LSPGK |
| 6 | Engineered | MGWSCIILFLVATATGVHSPKSCDKTHTCPPCPAPELLGGP |
| | Fc-IL-15 | SVFLFPPKPKDTLMISRTPEVTCVVVDVSHEDPEVKFNWY |
| | | VDGVEVHNAKTKPREEQYNSTYRVVSVLTVLHQDWLNG |
| | | KEYKCKVSNKALPAPIEKTISKAKGQPREPQVYTLPPSRDE |
| | | LTKNQVSLTCLVKGFYPSDIAVEWESNGQPENNYKTTPPV |
| | | LDSDGSFFLYSKLTVDKSRWQQGNVFSCSVMHEALHNHY |
| | | TQKSLSLSPGKNWVNVISDLKKIEDLIQSMHIDATLYTESD |
| | | VHPSCKVTAMKCFLLELQVISLESGDASIHDTVENLIILAN |
| | | NSLSSNGNVTESGCKECEELEEKNIKEFLQSFVHIVQMFIN |
| | | TS |
| 7 | Engineered | MGWSCIILFLVATATGVHSNWVNVISDLKKIEDLIQSMHI |
| | IL-15-Fc | DATLYTESDVHPSCKVTAMKCFLLELQVISLESGDASIHD |
| | | TVENLIILANNSLSSNGNVTESGCKECEELEEKNIKEFLQSF |
| | | VHIVQMFINTSPKSCDKTHTCPPCPAPELLGGPSVFLFPPKP |
| | | KDTLMISRTPEVTCVVVDVSHEDPEVKFNWYVDGVEVHN |
| | | AKTKPREEQYNSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFF |
| | | LYSKLTVDKSRWQQGNVFSCSVMHEALHNHYTQKSLSLS |
| | | PGK |

The protein cage polypeptide of the CCC can comprise a polypeptide of between about 400 and about 700 amino acid residues in length. In some embodiments, the protein cage polypeptide can comprise a polypeptide of about 450 to about 650 amino acid residues in length.

In some embodiments, the protein cage polypeptide can be comprised of a polypeptide comprising an amino acid sequence with at least about 70%, 75%, 80%, 85%, 90%, 95%, 99%, or 100% amino acid identity to the amino acid sequence set forth in any one of SEQ ID NOS:8-11 (see Table 2).

The protein cage polypeptide set forth in SEQ ID NO.12 can be designed by the applicant to be a Y294A mutant of the protein cage polypeptide set forth in SEQ ID NO.8 (see Table 2). The protein cage polypeptide set forth in SEQ ID NO.13 can be designed by the applicant to be a Y294A mutant of the protein cage polypeptide set forth in SEQ ID NO.9 (see Table 2). The protein cage polypeptide set forth in SEQ ID NO.14 can be designed by the applicant to be a Y294A mutant of the protein cage polypeptide set forth in SEQ ID NO.10 (see Table 2). The protein cage polypeptide set forth in SEQ ID NO.15 can be designed by the applicant to be a Y294A mutant of the protein cage polypeptide set forth in SEQ ID NO:11 (see Table 2).

The various protein cage polypeptides disclosed herein can also comprise peptide sequences capable of binding to the engineered Fc antibody domain. For example, the protein cage polypeptides set forth in SEQ ID NOS: 8 and 12 can comprise the amino acid sequence RWGSGADCAWHLGELVWCTAGSGWE (SEQ ID NO:16) (referred to herein as Peptide Sequence A, see Table 2) for binding to the engineered Fc antibody domain.

In addition, the protein cage polypeptides set forth in SEQ ID NOS: 9 and 13 can comprise the amino acid sequence GGRWGADCAWHLGELVWCTAGWEGG (SEQ ID NO: 17) (referred to herein as Peptide Sequence B, see Table 2) for binding to the engineered Fc antibody domain.

Moreover, the protein cage polypeptides set forth in SEQ ID NOS: 10 and 14 can comprise the amino acid sequence GADCAWHLGELVWCTAG (SEQ ID NO: 18) (referred to herein as Peptide Sequence C, see Table 2) for binding to the engineered Fc antibody domain.

Furthermore, the protein cage polypeptides set forth in SEQ ID NOS: 11 and 15 can comprise the amino acid sequence RWGSGCDCAWHLGELVWCTCGSGWE (SEQ ID NO: 19) (referred to herein as Peptide Sequence D, see Table 2) for binding to the engineered Fc antibody domain.

TABLE 2
Sequences of protein cage polypeptide variants designed and
experimentally tested to-date and peptide sequences capable
of binding to the engineered Fc antibody domain.

| SEQ ID NO: | NAME | SEQUENCE |
|---|---|---|
| 8 | Protein cage | MPFITVGQENSTSIDLYYEDHGTGTPVVLIHGFPLSGHSWE |
| | polypeptide | RQSAALLDAGYRVITYDRRGFGQSSQPTTGYDYDTFAAD |
| | 1 | LNTVLETLDLQDAVLVGFSMGTGEVARYVSSYGTARIAA |
| | | VAFLASLEPFLLKTDDNPDGAAPQEFFDGIVAAVKADRYA |
| | | FYTGFFNDFYNLDENLGTRISEEAVRNSWNTAASGGFFAA |
| | | AAAPTTWYTDFRADIPRIDVPALILHGTGDRTLPIENTARV |
| | | FHKALPSAEYVEVEGAPHGLLWTHAEEVNTALLAFLAKA |
| | | QEAQKQKLLTEVETYVLSIIPSGPLKAEIAQRLEDVFAGR |
| | | WGSGADCAWHLGELVWCTAGSGWEDLEVLMEWLKTRP |
| | | ILSPLTKGILGFVFTLTVPSERGLQRRRFVQNALNGNGDPN |
| | | NMDKAVKLYRKLKREITFHGAKEISLSYSAGALASCMGLI |
| | | YNRMGAVTTEVAFGLVCATCEQIADSQHRSHRQLEHHHH |
| | | HH |
| 9 | Protein cage | MPFITVGQENSTSIDLYYEDHGTGTPVVLIHGFPLSGHSWE |
| | polypeptide | RQSAALLDAGYRVITYDRRGFGQSSQPTTGYDYDTFAAD |
| | 2 | LNTVLETLDLQDAVLVGFSMGTGEVARYVSSYGTARIAA |
| | | VAFLASLEPFLLKTDDNPDGAAPQEFFDGIVAAVKADRYA |
| | | FYTGFFNDFYNLDENLGTRISEEAVRNSWNTAASGGFFAA |
| | | AAAPTTWYTDFRADIPRIDVPALILHGTGDRTLPIENTARV |
| | | FHKALPSAEYVEVEGAPHGLLWTHAEEVNTALLAFLAKA |
| | | QEAQKQKLLTEVETYVLSIIPSGPLKAEIAQRLEDVFAGGG |
| | | RWGADCAWHLGELVWCTAGWEGGDLEVLMEWLKTRPI |
| | | LSPLTKGILGFVFTLTVPSERGLQRRRFVQNALNGNGDPN |
| | | NMDKAVKLYRKLKREITFHGAKEISLSYSAGALASCMGLI |
| | | YNRMGAVTTEVAFGLVCATCEQIADSQHRSHRQLEHHHH |
| | | HH |
| 10 | Protein cage | MPFITVGQENSTSIDLYYEDHGTGTPVVLIHGFPLSGHSWE |
| | polypeptide | RQSAALLDAGYRVITYDRRGFGQSSQPTTGYDYDTFAAD |
| | 3 | LNTVLETLDLQDAVLVGFSMGTGEVARYVSSYGTARIAA |
| | | VAFLASLEPFLLKTDDNPDGAAPQEFFDGIVAAVKADRYA |
| | | FYTGFFNDFYNLDENLGTRISEEAVRNSWNTAASGGFFAA |
| | | AAAPTTWYTDFRADIPRIDVPALILHGTGDRTLPIENTARV |
| | | FHKALPSAEYVEVEGAPHGLLWTHAEEVNTALLAFLAKA |
| | | QEAQKQKLLTEVETYVLSIIPSGPLKAEIAQRLEDVFAGGA |
| | | DCAWHLGELVWCTAGDLEVLMEWLKTRPILSPLTKGILG |
| | | FVFTLTVPSERGLQRRRFVQNALNGNGDPNNMDKAVKLY |
| | | RKLKREITFHGAKEISLSYSAGALASCMGLIYNRMGAVTT |
| | | EVAFGLVCATCEQIADSQHRSHRQLEHHHHHH |
| 11 | Protein cage | MPFITVGQENSTSIDLYYEDHGTGTPVVLIHGFPLSGHSWE |
| | polypeptide | RQSAALLDAGYRVITYDRRGFGQSSQPTTGYDYDTFAAD |
| | 4 | LNTVLETLDLQDAVLVGFSMGTGEVARYVSSYGTARIAA |
| | | VAFLASLEPFLLKTDDNPDGAAPQEFFDGIVAAVKADRYA |
| | | FYTGFFNDFYNLDENLGTRISEEAVRNSWNTAASGGFFAA |
| | | AAAPTTWYTDFRADIPRIDVPALILHGTGDRTLPIENTARV |
| | | FHKALPSAEYVEVEGAPHGLLWTHAEEVNTALLAFLAKA |
| | | QEAQKQKLLTEVETYVLSIIPSGPLKAEIAQRLEDVFAGR |
| | | WGSGCDCAWHLGELVWCTCGSGWEDLEVLMEWLKTRPI |
| | | LSPLTKGILGFVFTLTVPSERGLQRRRFVQNALNGNGDPN |
| | | NMDKAVKLYRKLKREITFHGAKEISLSYSAGALASCMGLI |
| | | YNRMGAVTTEVAFGLVCATCEQIADSQHRSHRQLEHHHH |
| | | HH |
| 12 | Protein cage | MPFITVGQENSTSIDLYYEDHGTGTPVVLIHGFPLSGHSWE |
| | polypeptide | RQSAALLDAGYRVITYDRRGFGQSSQPTTGYDYDTFAAD |
| | 5 | LNTVLETLDLQDAVLVGFSMGTGEVARYVSSYGTARIAA |
| | | VAFLASLEPFLLKTDDNPDGAAPQEFFDGIVAAVKADRYA |
| | | FYTGFFNDFYNLDENLGTRISEEAVRNSWNTAASGGFFAA |
| | | AAAPTTWYTDFRADIPRIDVPALILHGTGDRTLPIENTARV |
| | | FHKALPSAEYVEVEGAPHGLLWTHAEEVNTALLAFLAKA |
| | | QEAQKQKLLTEVETAVLSIIPSGPLKAEIAQRLEDVFAGR |
| | | WGSGADCAWHLGELVWCTAGSGWEDLEVLMEWLKTRP |
| | | ILSPLTKGILGFVFTLTVPSERGLQRRRFVQNALNGNGDPN |
| | | NMDKAVKLYRKLKREITFHGAKEISLSYSAGALASCMGLI |
| | | YNRMGAVTTEVAFGLVCATCEQIADSQHRSHRQLEHHHH |
| | | HH |
| 13 | Protein cage | MPFITVGQENSTSIDLYYEDHGTGTPVVLIHGFPLSGHSWE |
| | polypeptide | RQSAALLDAGYRVITYDRRGFGQSSQPTTGYDYDTFAAD |
| | 6 | LNTVLETLDLQDAVLVGFSMGTGEVARYVSSYGTARIAA |
| | | VAFLASLEPFLLKTDDNPDGAAPQEFFDGIVAAVKADRYA |
| | | FYTGFFNDFYNLDENLGTRISEEAVRNSWNTAASGGFFAA |
| | | AAAPTTWYTDFRADIPRIDVPALILHGTGDRTLPIENTARV |
| | | FHKALPSAEYVEVEGAPHGLLWTHAEEVNTALLAFLAKA |
| | | QEAQKQKLLTEVETAVLSIIPSGPLKAEIAQRLEDVFAGGG |
| | | RWGADCAWHLGELVWCTAGWEGGDLEVLMEWLKTRPI |
| | | LSPLTKGILGFVFTLTVPSERGLQRRRFVQNALNGNGDPN |
| | | NMDKAVKLYRKLKREITFHGAKEISLSYSAGALASCMGLI |
| | | YNRMGAVTTEVAFGLVCATCEQIADSQHRSHRQLEHHHH |
| | | HH |
| 14 | Protein cage | MPFITVGQENSTSIDLYYEDHGTGTPVVLIHGFPLSGHSWE |
| | polypeptide | RQSAALLDAGYRVITYDRRGFGQSSQPTTGYDYDTFAAD |
| | 7 | LNTVLETLDLQDAVLVGFSMGTGEVARYVSSYGTARIAA |
| | | VAFLASLEPFLLKTDDNPDGAAPQEFFDGIVAAVKADRYA |
| | | FYTGFFNDFYNLDENLGTRISEEAVRNSWNTAASGGFFAA |
| | | AAAPTTWYTDFRADIPRIDVPALILHGTGDRTLPIENTARV |
| | | FHKALPSAEYVEVEGAPHGLLWTHAEEVNTALLAFLAKA |
| | | QEAQKQKLLTEVETAVLSIIPSGPLKAEIAQRLEDVFAGGA |
| | | DCAWHLGELVWCTAGDLEVLMEWLKTRPILSPLTKGILG |
| | | FVFTLTVPSERGLQRRRFVQNALNGNGDPNNMDKAVKLY |
| | | RKLKREITFHGAKEISLSYSAGALASCMGLIYNRMGAVTT |
| | | EVAFGLVCATCEQIADSQHRSHRQLEHHHHHH |
| 15 | Protein cage | MPFITVGQENSTSIDLYYEDHGTGTPVVLIHGFPLSGHSWE |
| | polypeptide | RQSAALLDAGYRVITYDRRGFGQSSQPTTGYDYDTFAAD |
| | 8 | LNTVLETLDLQDAVLVGFSMGTGEVARYVSSYGTARIAA |
| | | VAFLASLEPFLLKTDDNPDGAAPQEFFDGIVAAVKADRYA |
| | | FYTGFFNDFYNLDENLGTRISEEAVRNSWNTAASGGFFAA |
| | | AAAPTTWYTDFRADIPRIDVPALILHGTGDRTLPIENTARV |
| | | FHKALPSAEYVEVEGAPHGLLWTHAEEVNTALLAFLAKA |
| | | QEAQKQKLLTEVETAVLSIIPSGPLKAEIAQRLEDVFAGR |
| | | WGSGCDCAWHLGELVWCTCGSGWEDLEVLMEWLKTRPI |
| | | LSPLTKGILGFVFTLTVPSERGLQRRRFVQNALNGNGDPN |
| | | NMDKAVKLYRKLKREITFHGAKEISLSYSAGALASCMGLI |
| | | YNRMGAVTTEVAFGLVCATCEQIADSQHRSHRQLEHHHH |
| | | HH |
| 16 | Peptide | RWGSGADCAWHLGELVWCTAGSGWE |
| | sequence A, | |
| | capable of | |
| | binding to the | |
| | engineered | |
| | Fc antibody | |
| | domain | |
| 17 | Peptide | GGRWGADCAWHLGELVWCTAGWEGG |
| | sequence B, | |
| | capable of | |
| | binding to the | |
| | engineered | |
| | Fc antibody | |
| | domain | |
| 18 | Peptide | GADCAWHLGELVWCTAG |
| | sequence C, | |
| | capable of | |
| | binding to the | |
| | engineered | |
| | Fc antibody | |
| | domain | |
| 19 | Peptide | RWGSGCDCAWHLGELVWCTCGSGWE |
| | sequence D, | |
| | capable of | |
| | binding to the | |
| | engineered | |
| | Fc antibody | |
| | domain | |

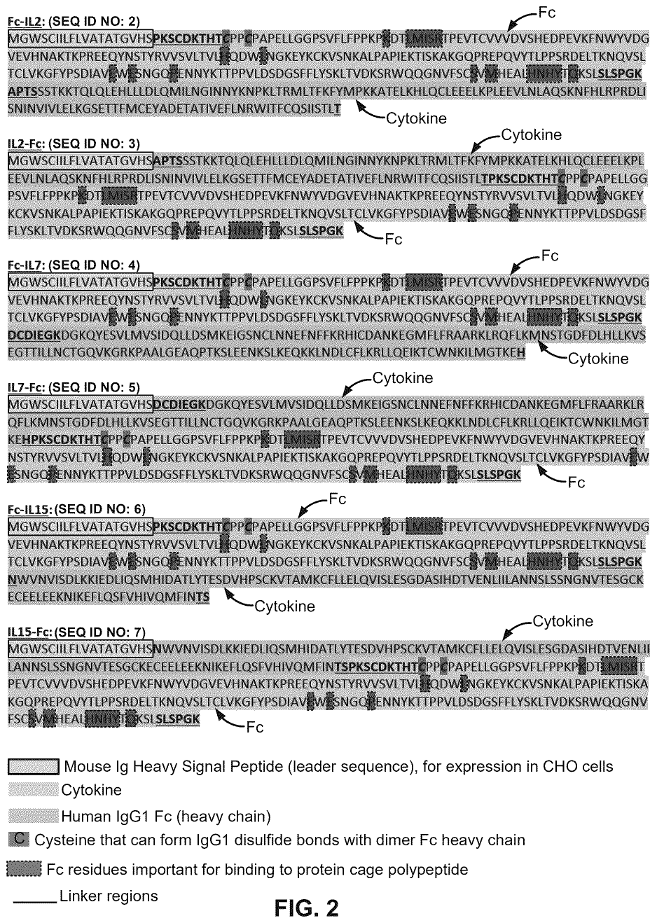

FIG. 2 illustrates that the engineered Fc antibody domains can be C-terminally linked or N-terminally linked to the cytokines (the Fc-cytokines can then be bound to a protein cage polypeptide) to achieve their augmented function of activating and expanding T cells beyond the capabilities of soluble cytokines alone. Although the applicant anticipates that C-terminally linked Fc-cytokines facilitate less sterically-restricted FcγR binding, one unexpected discovery made by the applicant is that N-terminally linked Fc-cytokine CCCs also work well to activate and expand T cells beyond the capabilities of soluble cytokines.

As shown in FIG. 2, the engineered Fc antibody domains of the CCCs can be C-terminally linked to an N-terminus of at least one of the cytokines. Table 1 lists out representative sequences for several Fc-cytokine variants.

For example, when one of the cytokines is interleukin-2 (IL-2), at least one of the engineered Fc antibody domains can be C-terminally linked to the N-terminus of the IL-2 (herein referred to as Fc-IL-2) via an engineered metalloprotease-resistant linker sequence. In some embodiments, the engineered metalloprotease-resistant linker sequence can comprise the amino acid sequence SLSPGKAPTS (SEQ ID NO:20) (see, also, FIG. 3).

Also, for example, when one of the cytokines is interleukin-7 (IL-7), at least one of the engineered Fc antibody domains can be C-terminally linked to the N-terminus of the IL-7 (herein referred to as Fc-IL-7) via an engineered metalloprotease-resistant linker sequence. In some embodiments, the engineered metalloprotease-resistant linker sequence can comprise the amino acid sequence SLSPGKDCDIEGK (SEQ ID NO:21) (see, also, FIG. 3).

As an additional example, when one of the cytokines is interleukin-15 (IL-15), at least one of the engineered Fc antibody domains can be C-terminally linked to the N-terminus of the IL-15 (herein referred to as Fc-IL-15) via an engineered metalloprotease-resistant linker sequence. In some embodiments, the engineered metalloprotease-resistant linker sequence can comprise the amino acid sequence SLSPGKN (SEQ ID NO:22) (see, also, FIG. 3).

FIG. 2 also illustrates that the engineered Fc antibody domains can be N-terminally linked to a C-terminus of at least one of the cytokines (which can then be bound to a protein cage polypeptide) to achieve their augmented function of activating and expanding T cells beyond the capabilities of soluble cytokines alone. Table 1 also lists out representative sequences for these Fc-cytokine variants.

For example, when one of the cytokines is interleukin-2 (IL-2), at least one of the engineered Fc antibody domains can be N-terminally linked to the C-terminus of the IL-2 (herein referred to as IL-2-Fc) via an engineered metalloprotease-resistant linker sequence. In some embodiments, the engineered metalloprotease-resistant linker can comprise the amino acid sequence TPKSCDKTHT (SEQ ID NO:23) (see, also, FIG. 3).

Also, for example, when one of the cytokines is interleukin-7 (IL-7), at least one of the engineered Fc antibody domains can be N-terminally linked to the C-terminus of the IL-7 (herein referred to as Fc-IL-7) via an engineered metalloprotease-resistant linker sequence. In some embodiments, the engineered metalloprotease-resistant linker sequence can comprise the amino acid sequence HPKSCDKTHT (SEQ ID NO:24) (see, also, FIG. 3).

As an additional example, when one of the cytokines is interleukin-15 (IL-15), at least one of the engineered Fc antibody domains can be N-terminally linked to the C-terminus of the IL-15 (herein referred to as Fc-IL-15) via an engineered metalloprotease-resistant linker sequence. In some embodiments, the engineered metalloprotease-resistant linker sequence can comprise the amino acid sequence TSPKSCDKTHT (SEQ ID NO:25) (see, also, FIG. 3).

FIG. 2 also illustrates that the CCCs can comprise a signal peptide or leader sequence linked to an N-terminus of at least one of the engineered Fc antibody domains or at least one of the cytokines.

In some embodiments, the signal peptide can be a murine Ig heavy signal peptide for expression in Chinese hamster ovary (CHO) cells. The signal peptide can improve the secretion efficiency of the engineered molecule in CHO cells.

As a more specific example, the signal peptide can comprise the amino acid sequence MGWSCIILFLVATATGVHS (SEQ ID NO:26).

FIG. 3 illustrates example linker sequences designed to link the Fc and cytokine components of the following CCCs: (1) Fc-IL-2 CCC, (2) IL-2-Fc CCC, (3) Fc-IL-7 CCC, (4) IL-7-Fc CCC, (5) Fc-IL-15 CCC, and (6) IL-15-Fc CCC (see, also, Table 1). The linkers are underlined in FIG. 3.

Also shown in FIG. 3 are various cleavage sites (e.g., for aspartic protease, cysteine protease, metalloprotease, serine protease, and different multiple protease superfamilies). As will be discussed in more detail with respect to Example 3, metalloproteases are known for mediating cell-surface receptor shedding or cleaving the functional domains of receptors and ligands off of the cells. FIG. 3 shows that none of the linkers are cleaved in the middle of the linkers except for serine proteases. Serine proteases are less of a concern since they are often localized inside the cells themselves.

The amino acid sequences shown in FIG. 3 (and listed below) are portions of the amino acid sequences listed in Table 1 above:

Portion of Engineered Fc-IL-2:
(SEQ ID NO: 27)
QQGNVFSCSVMHEALHNHYTQKSLSLSPGKAPTSSSTKKTQLQLEHLLL
DLQMILNGINNYKNPKLTRML.
Portion of Engineered IL-2-Fc:
(SEQ ID NO: 28)
WITFCQSIISTLTPKSCDKTHTCPPCPAPELLGGPSVFLFPPKPKDTLM
ISRTP.
Portion of Engineered Fc-IL-7:
(SEQ ID NO: 29)
SRWQQGNVFSCSVMHEALHNHYTQKSLSLSPGKDCDIEGKDGKQYESVL
MVSIDQLLDSMKEIGSNCLNN.
Portion of Engineered IL-7-Fc:
(SEQ ID NO: 30)
DLCFLKRLLQEIKTCWNKILMGTKEHPKSCDKTHTCPPCPAPELLGGPS
VFLFPPKPKDTLMISRTPEVTCVVV.
Portion of Engineered Fc-IL-15:
(SEQ ID NO: 31)
WQQGNVFSCSVMHEALHNHYTQKSLSLSPGKNWVNVISDLKKIEDLIQS
MHIDATLYTESDVHPSCKV.
Portion of Engineered IL-15-Fc:
(SEQ ID NO: 32)
FVHIVQMFINTSPKSCDKTHTCPPCPAPELLGGPSVFLFPPKP.

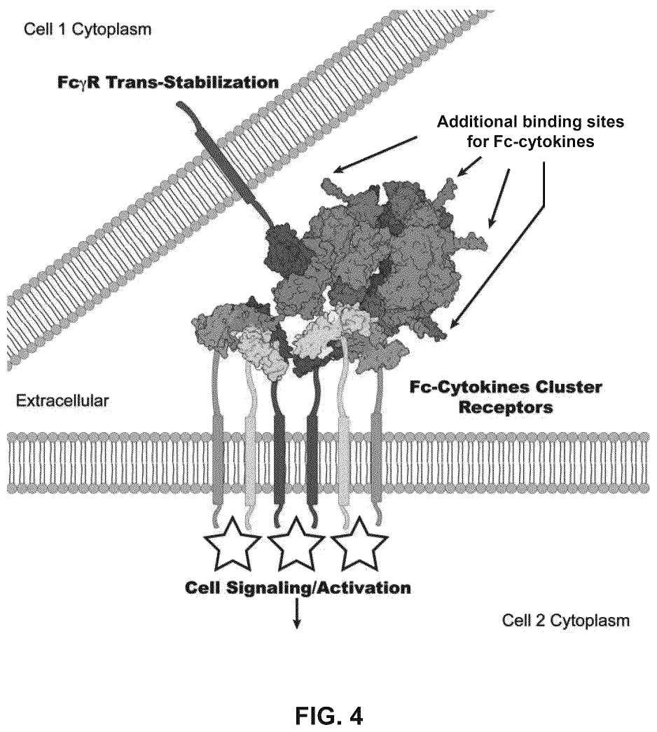

FIG. 4 illustrates an example of the engineered Fc component of the CCC stabilized by an Fcγ receptor (FcγR) of a nearby cell. The Fc components of the CCCs can be stabilized via trans-stabilization by nearby cells expressing FcγRs.

In some embodiments, the Fc component of the CCC can be engineered to comprise one or more point mutations to modulate FcγR binding. For example, the Fc component can be engineered to be either FcγR binding competent (i.e., increased affinity for FcγR binding) or FcγR binding incompetent (i.e., decreased affinity for FcγR binding) to avoid antibody-dependent cellular cytotoxicity (ADCC) activation of NK cells.

Three main classes of Fcγ receptors (FcγRs) exist on leukocytes (FcγRI, FcγRII, and FcγRIII) and function in cellular processes such as: ADCC, cytokine release, phagocytosis, and endocytosis [7]. A primary signal to cause NK cells to initiate killing activities, such as release of perforins and granzymes, is ADCC, which requires clustering of FcγRIIIa receptors on the NK cells' surface [8]. Depending on the application, NK cell-mediated ADCC may or may not be desirable.

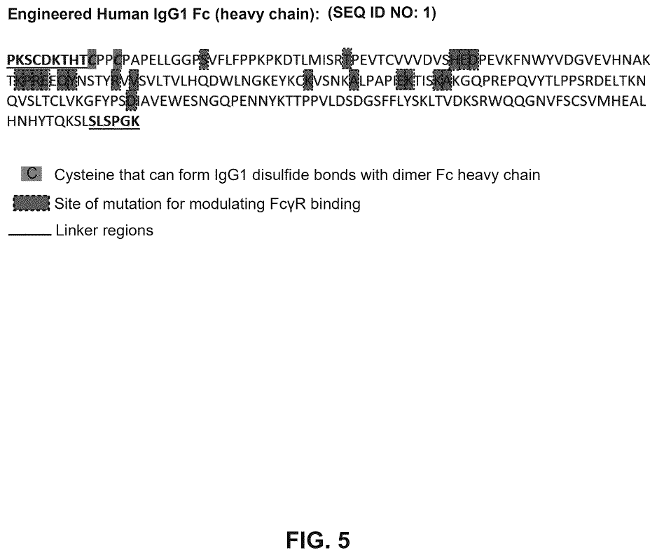

FIG. 5 illustrates that the Fc component (e.g., human IgG1 Fc) of the CCC can be engineered through mutation of key sites in its FcγR-binding portion. The various ways that the FcγR-binding portion can be modified via point mutations are shown in FIG. 5. Some such point mutations are also discussed in the literature [8, 9, and 10].

In some embodiments, the engineered human IgG1 Fc antibody domain can comprise at least one of the following point mutations relative to SEQ ID NO:1 (see Table 1) to decrease affinity for certain Fc receptors (e.g., FcγRIII) and functionally reduce ADCC: P75L, R76W, Y80K, Y80P, Y80R, Y80G, and Y80A.

For example, the Y80K variant can markedly reduce binding to the FcγRIIIa receptor and diminish ADCC. Also, for example, the Y80P variant can markedly reduce binding to FcγRIIIa and cause insufficient N-glycosylation.

As another example, the Y80R variant can markedly reduce binding to the FcγRIIIa receptor. Also, for example, the Y80G variant can markedly reduce binding to the FcγRIIIa receptor. As yet another example, the Y80A variant can markedly reduce binding to the FcγRIIIa receptor and diminish ADCC.

In some embodiments, the engineered human IgG1 Fc antibody domain can comprise at least one of the following point mutations relative to SEQ ID NO:1 (see Table 1) to increase affinity for certain Fcγ receptors and functionally increase ADCC: Y80W.

In some embodiments, the engineered human IgG1 Fc antibody domain can comprise at least one of the following point mutations relative to SEQ ID NO:1 (see Table 1) to decrease affinity for certain Fcγ receptors (e.g., FcγRIIIa) and have a neutral effect on other Fcγ receptors (e.g., FcγRII): S23A, E53A, E77A, Y80F, V87A, A111G, K122A, and D160A.

In some embodiments, the engineered human IgG1 Fc antibody domain can comprise at least one of the following point mutations relative to SEQ ID NO:1 (see Table 1) to increase affinity for certain Fcγ receptors (e.g., FcγRIIIa) and have a neutral effect on other Fcγ receptors (e.g., FcγRII): E117, K118A, and A123T.

In some embodiments, the engineered human IgG1 Fc antibody domain can comprise at least one of the following point mutations relative to SEQ ID NO:1 (see Table 1) to increase affinity for certain Fcγ receptors (e.g., FcγRII) and decrease affinity for other Fcγ receptors (e.g., FcγRIIIa): H52A, R85A, and K106A.

In some embodiments, the engineered human IgG1 Fc antibody domain can comprise at least one of the following point mutations relative to SEQ ID NO:1 (see Table 1) to decrease affinity for certain Fcγ receptors (e.g., FcγRII and FcγRIIIa): D54A, Q79A, and A111S.

In some embodiments, the engineered human IgG1 Fc antibody domain can comprise at least one of the following point mutations relative to SEQ ID NO:1 (see Table 1) to increase affinity for certain Fcγ receptors (e.g., FcγRII and FcγRIIIa): T40A and K74A.

As previously discussed, the Fc components of the CCCs can be engineered to be either FcγR binding competent (i.e., increased affinity for FcγR binding) to functionally increase ADCC or FcγR binding incompetent (i.e., decreased affinity for FcγR binding) to functionally decrease ADCC.

### EXAMPLES

The examples below are given so as to illustrate the practice of various embodiments of the present disclosure. They are not intended to limit or define the entire scope of this disclosure. It should be appreciated that the disclosure is not limited to the particular embodiments described and illustrated herein but includes all modifications and variations falling within the scope of the disclosure as defined in the appended embodiments.

### Example 1: Chimeric Cytokine Complexes (CCCs) Enhance T-Cell Activation and Expansion Beyond Soluble Cytokine Capabilities

One unexpected result stemming from the experiments disclosed herein is that CCCs enhance or markedly increase T-cell activation and expansion than equivalent concentrations of soluble cytokines alone or even Fc-cytokines alone.

Human peripheral blood CD3⁺ T cells were thawed and media exchanged in serum free T-cell expansion medium. T cells were seeded at a concentration of 1×10⁶ cells/mL then placed in a 37° C. and 5% CO₂ incubator on day −1. On day 0, cells were activated with an anti-CD3 and anti-CD28 T-cell activation reagent. In some embodiments, the T-cell activation reagent can be a soluble T-cell activator for use in vitro.

In some embodiments, the anti-CD3 and anti-CD28 T-cell activation reagent can comprise a plurality of self-assembling protein nanoparticles decorated with anti-CD3 antibodies and anti-CD28 antibodies. The self-assembling protein nanoparticles can comprise protein cage polypeptides assembled into three-dimensional structures.

In some embodiments, the three-dimensional structure of the protein cage polypeptide can be a tetrahedral pyramid. The protein cage polypeptides can also self-assemble into compact asymmetrical multimeric structures or cage-cage multimers (including dimers).

The three-dimensional structures (e.g., protein cage polypeptide formed into tetrahedral pyramids) can serve as scaffolds for the anti-CD3 antibodies and the anti-CD28 antibodies.

In some embodiments, the protein cage polypeptide can be comprised of a polypeptide comprising an amino acid sequence with at least about 70%, 75%, 80%, 85%, 90%, 95%, 99%, or 100% amino acid identity to the amino acid sequence set forth in any one of SEQ ID NOS: 8-10 (see Table 2).

The protein cage polypeptide can comprise a polypeptide of between about 400 and about 700 amino acid residues in length. In some embodiments, the protein cage polypeptide can comprise a polypeptide of about 450 amino acid residues to about 650 amino acid residues in length.

In some embodiments, the anti-CD3 antibodies can be the “OKT3” clone with multiple host isotypes, such as human, mouse, rabbit, etc. For example, any of the following anti-CD3 antibodies can be used: (i) anti-CD3 monoclonal antibodies (OKT3) distributed by Takara Bio, (ii) GMP monoclonal anti-human CD3 antibodies (OKT3) distributed by ACROBiosystems, (iii) MACS® GMP CD3 pure antibodies distributed by Miltenyi Biotec, or (iv) GMP Ultra-LEAF™ purified anti-human CD3 SF antibodies distributed by BioLegend.

In some embodiments, the anti-CD28 antibodies can be agonist clones with multiple host isotypes, such as human, mouse, rabbit, etc. For example, any of the following anti-CD28 antibodies can be used: (i) anti-CD28 [YTH 913.12] antibodies distributed by Absolute Antibody, (ii) CD28 antibodies, anti-human, clone 15E8 distributed by Miltenyi Biotec, (iii) Ultra-LEAF™ purified anti-human CD28 antibodies, clone cd28.2, distributed by BioLegend, or (iv) BD™ purified mouse anti-human CD28 antibodies, clone L293, distributed by BD Biosciences.

Standard soluble cytokines were added to the medium at concentrations of 10 ng/mL, 15 ng/mL, 20 ng/mL, 25 ng/mL, 30 ng/mL, and 35 ng/mL and CCCs were added to the medium at concentrations equivalent (molar equivalent, m.e.) to those of the standard cytokines. 10 ng/mL of Fc-IL-2s were also added. The CCCs added were FC-IL-2 CCCs. The cytokines added were soluble IL-2s. Cells were then returned to the 37° C. and 5% CO₂ incubator.

On day 5, medium was collected for an ELISA specific for detecting interferon-gamma (IFN-7). FIG. 6 is a graph illustrating the results of the ELISA specific for detecting IFN-7. As shown in FIG. 6, T cells on media supplemented with CCCs (e.g., the FC-IL-2 CCCs) were activated much more than T cells on media supplemented with cytokines (e.g., IL-2s) alone or even Fc-cytokines (e.g., Fc-IL-2s) as indicated by levels of IFN-7 produced by the T cells.

Fresh cell culture medium supplemented with either CCC or standard cytokines was added to the cells at equivalent concentrations (e.g., 10 ng/mL, 15 ng/mL, 20 ng/mL, 25 ng/mL, 30 ng/mL, and 35 ng/mL). Cells were then returned to the 37° C. and 5% CO₂ incubator. On day 7, cells were resuspended and collected for flow cytometry.

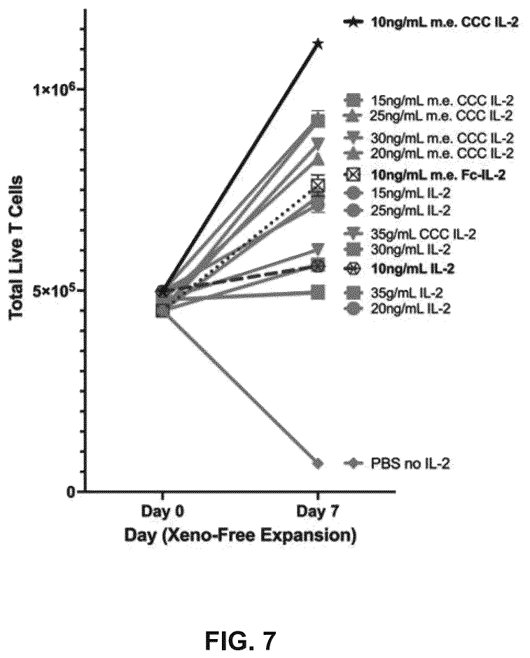

FIG. 7 is a graph illustrating the population of live T cells as determined using flow cytometry on day 7. Cells were stained with Zombie Aqua to determine the population of live T cells. As shown in FIG. 7, total live T cells on media supplemented with CCCs (e.g., the Fc-IL-2 CCCs) greatly exceeded total live T cells on media supplemented with cytokines (e.g., IL-2s) alone or even Fc-cytokines (e.g., Fc-IL-2s) on day 7.

One unexpected result stemming from the experiments disclosed herein is that CCCs at concentrations between 10 ng/mL and 50 ng/mL of media significantly enhance T-cell expansion than equivalent and even greater concentrations of soluble cytokines (e.g., IL-2s) alone.

Another unexpected result stemming from the experiments disclosed herein is that Fc-cytokine complexes (e.g., Fc-IL-2s) of the type disclosed herein also enhance T-cell expansion than equivalent and even greater concentrations of soluble cytokines (e.g., IL-2s) alone.

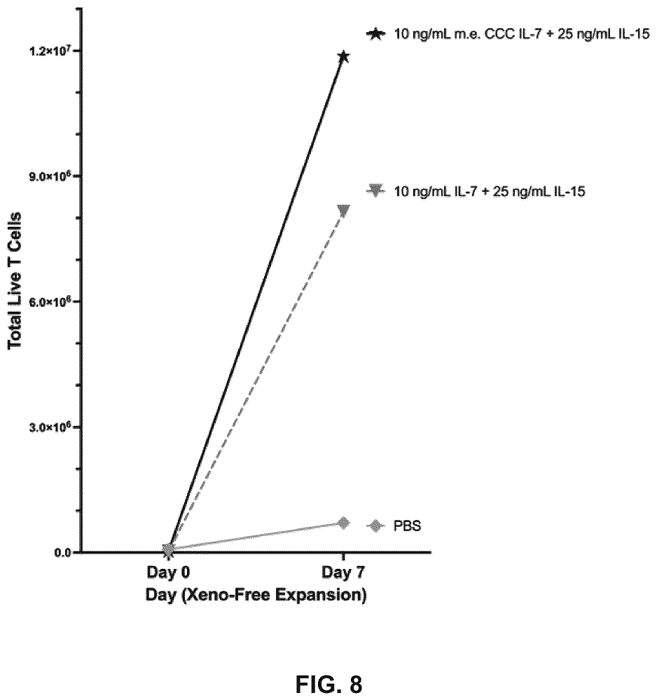

FIG. 8 is a graph illustrating the population of live T cells as determined using flow cytometry on day 7. Cells were stained with Zombie Aqua to determine the population of live T cells. As shown in FIG. 8, total live T cells on media supplemented with a combination of IL-7-Fc CCCs (i.e., wherein the engineered Fc antibody domain is N-terminally linked to the C-terminus of IL-7 cytokine) and soluble cytokines (e.g., IL-15s) greatly exceeded total live T cells on media supplemented with only soluble cytokines (e.g., IL-7s and IL-15s) on day 7.

### Example 2: Chimeric Cytokine Complexes (CCCs) Increase CD4⁺ T Cell Count in Expanded T Cells Beyond Soluble Cytokine Capabilities

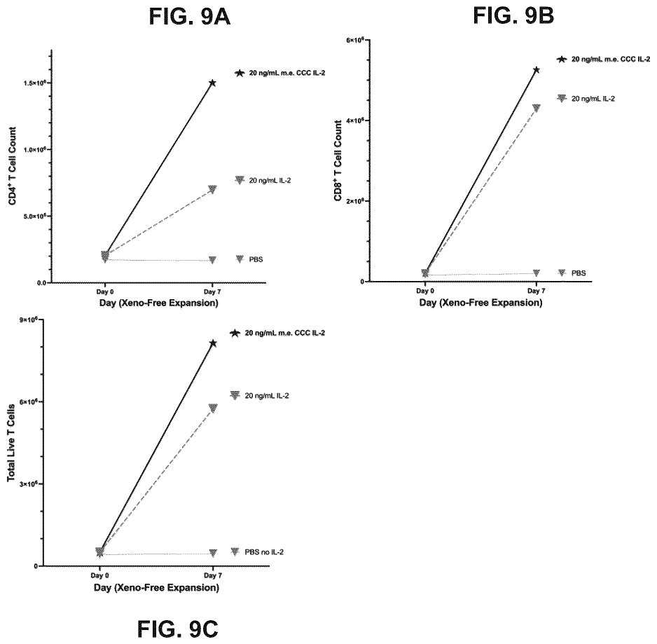

Yet another unexpected result stemming from the experiments disclosed herein is that CCCs greatly increase the CD4⁺ content of the expanded T cells. FIGS. 9A-9C are graphs illustrating the CD4⁺ T cell count, the CD8⁺ T cell count, and the total live T cells at day 7, respectively.

Human peripheral blood T cells were thawed and media exchanged in serum free T-cell expansion medium. T cells were seeded at a concentration of 1×10⁶ cells/mL then placed in a 37° C. and 5% CO₂ incubator on day −1. On day 0, cells were activated with an anti-CD3 and anti-CD28 T-cell activation reagent.

In some embodiments, the anti-CD3 and anti-CD28 T-cell activation reagent can comprise a plurality of self-assembling protein nanoparticles decorated with anti-CD3 antibodies and anti-CD28 antibodies. The self-assembling protein nanoparticles can comprise protein cage polypeptides assembled into three-dimensional structures.

In some embodiments, the three-dimensional structure of the protein cage polypeptide can be a tetrahedral pyramid. The protein cage polypeptides can also self-assemble into compact asymmetrical multimeric structures or cage-cage multimers (including dimers).

The three-dimensional structures (e.g., protein cage polypeptide formed into tetrahedral pyramids) can serve as scaffolds for the anti-CD3 antibodies and the anti-CD28 antibodies.

In some embodiments, the protein cage polypeptide can be comprised of a polypeptide comprising an amino acid sequence with at least about 70%, 75%, 80%, 85%, 90%, 95%, 99%, or 100% amino acid identity to the amino acid sequence set forth in any one of SEQ ID NOS: 8-10 (see Table 2).

The protein cage polypeptide can comprise a polypeptide of between about 400 and about 700 amino acid residues in length. In some embodiments, the protein cage polypeptide can comprise a polypeptide of about 450 amino acid residues to about 650 amino acid residues in length.

In some embodiments, the anti-CD3 antibodies can be the “OKT3” clone with multiple host isotypes, such as human, mouse, rabbit, etc. For example, any of the following anti-CD3 antibodies can be used: (i) anti-CD3 monoclonal antibodies (OKT3) distributed by Takara Bio, (ii) GMP monoclonal anti-human CD3 antibodies (OKT3) distributed by ACROBiosystems, (iii) MACS® GMP CD3 pure antibodies distributed by Miltenyi Biotec, or (iv) GMP Ultra-LEAF™ purified anti-human CD3 SF antibodies distributed by BioLegend.

In some embodiments, the anti-CD28 antibodies can be agonist clones with multiple host isotypes, such as human, mouse, rabbit, etc. For example, any of the following anti-CD28 antibodies can be used: (i) anti-CD28 [YTH 913.12] antibodies distributed by Absolute Antibody, (ii) CD28 antibodies, anti-human, clone 15E8 distributed by Miltenyi Biotec, (iii) Ultra-LEAF™ purified anti-human CD28 antibodies, clone cd28.2, distributed by BioLegend, or (iv) BD™ purified mouse anti-human CD28 antibodies, clone L293, distributed by BD Biosciences.

Standard soluble cytokines were added to the medium at a concentration of 20 ng/mL and CCCs were added to the medium at concentrations equivalent (molar equivalent, m.e.) to those of the standard cytokines. The CCCs added were IL-2-Fc CCCs. The cytokines added to compare were soluble IL-2s. On day 7, cells were resuspended and collected for flow cytometry. Cells were stained with Zombie Aqua to determine the population of live cells. The T-cells were then run on the flow cytometer.

### Example 3: Cytokine and Chemokine Multiplexing Reveal Increase in Expression of Matrix Metalloproteases (MMPs) by T Cells

Human peripheral blood CD3⁺ T cells were thawed and media exchanged in serum free T-cell expansion medium. T cells were seeded at a concentration of 1×10⁶ cells/mL in a GREX® 24-well plate then placed in a 37° C. and 5% CO₂ incubator on day −1. On day 0, T cells were activated with an anti-CD3 and anti-CD28 T-cell activation reagent.

In some embodiments, the anti-CD3 and anti-CD28 T-cell activation reagent can comprise a plurality of self-assembling protein nanoparticles decorated with anti-CD3 antibodies and anti-CD28 antibodies. The self-assembling protein nanoparticles can comprise protein cage polypeptides assembled into three-dimensional structures.

In some embodiments, the three-dimensional structure of the protein cage polypeptide can be a tetrahedral pyramid. The protein cage polypeptides can also self-assemble into compact asymmetrical multimeric structures or cage-cage multimers (including dimers).

The three-dimensional structures (e.g., protein cage polypeptide formed into tetrahedral pyramids) can serve as scaffolds for the anti-CD3 antibodies and the anti-CD28 antibodies.

In some embodiments, the protein cage polypeptide can be comprised of a polypeptide comprising an amino acid sequence with at least about 70%, 75%, 80%, 85%, 90%, 95%, 99%, or 100% amino acid identity to the amino acid sequence set forth in any one of SEQ ID NOS: 8-10 (see Table 2).

The protein cage polypeptide can comprise a polypeptide of between about 400 and about 700 amino acid residues in length. In some embodiments, the protein cage polypeptide can comprise a polypeptide of about 450 amino acid residues to about 650 amino acid residues in length.

In some embodiments, the anti-CD3 antibodies can be the “OKT3” clone with multiple host isotypes, such as human, mouse, rabbit, etc. For example, any of the following anti-CD3 antibodies can be used: (i) anti-CD3 monoclonal antibodies (OKT3) distributed by Takara Bio, (ii) GMP monoclonal anti-human CD3 antibodies (OKT3) distributed by ACROBiosystems, (iii) MACS® GMP CD3 pure antibodies distributed by Miltenyi Biotec, or (iv) GMP Ultra-LEAF™ purified anti-human CD3 SF antibodies distributed by BioLegend.

In some embodiments, the anti-CD28 antibodies can be agonist clones with multiple host isotypes, such as human, mouse, rabbit, etc. For example, any of the following anti-CD28 antibodies can be used: (i) anti-CD28 [YTH 913.12] antibodies distributed by Absolute Antibody, (ii) CD28 antibodies, anti-human, clone 15E8 distributed by Miltenyi Biotec, (iii) Ultra-LEAF™ purified anti-human CD28 antibodies, clone cd28.2, distributed by BioLegend, or (iv) BD™ purified mouse anti-human CD28 antibodies, clone L293, distributed by BD Biosciences.

Cytokines (e.g., IL-2s) were added to the medium to achieve a final concentration of 10 ng/mL. A 500 μL sample of medium without IL-2 was frozen and was the day 0 control sample. T cells were then returned to the 37° C. and 5% CO₂ incubator. On day 3, a 500 μL sample of medium was collected from each sample well and frozen. Fresh culture medium supplemented with IL-2 was then exchanged with spent media such that a final concentration of 10 ng/mL IL-2 was achieved. T cells were then returned to the 37° C. and 5% CO₂ incubator. On day 7, a 500 μL sample of medium was collected from each sample well and frozen. Fresh culture medium supplemented with IL-2 was then exchanged with spent media to achieve a final IL-2 concentration of 10 ng/mL. T cells were then returned to the 37° C. and 5% CO₂ incubator. The frozen day 0, day 3, and day 7 samples were subjected to a 71-human cytokine/chemokine multiplexing panel that uses a combination of antibody-coupled fluorescent beads to capture analytes and fluorescent detection antibodies to determine the concentration of cytokines and chemokines in the medium.

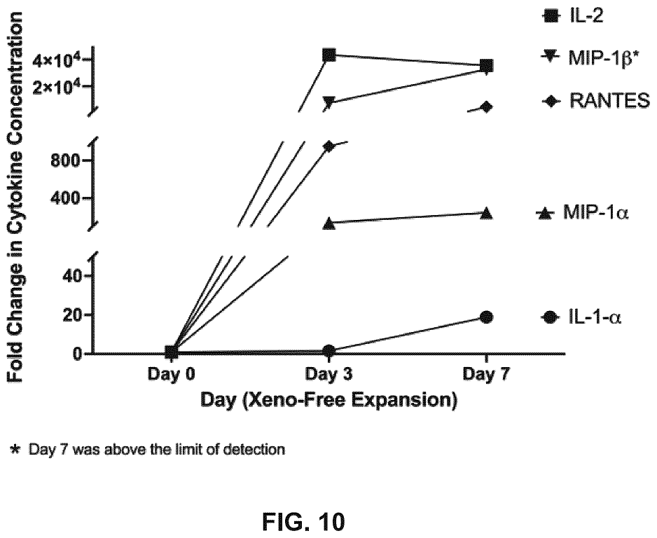

As shown by the graph in FIG. 10, upon stimulation with an anti-CD3 and anti-CD28 T-cell activation reagent, peripheral blood donor T cells upregulated the expression of cytokines and chemokines that increase the expression of matrix metalloproteases (MMPs) (also referred to as matrix metalloproteinases) by T cells.

This is in line with other research which showed that T cells and NK cells express MMPs and the receptor CD100 can be “shed’ from the surface through MMP proteolysis activity [11, 12]. Furthermore, it was shown that the chemokines and cytokines, TNF-alpha, IL-1, MIP-1alpha, MIP-1beta, and RANTES upregulated proMMP-9 (the pro-protease prior to enzymatic cleavage into active MMP-9) secretion by CD3⁺ and CD4⁺ T cells [13, 14]. Additionally, IL-2 stimulation increases MMP-9 production in T cells [11].

Connecting these lines of research with the pursuit of CCC-based and Fc-cytokine-based cellular agonism, there is a crucial need to protect the chimeric Fc-cytokine linkers of CCCs and Fc-cytokine complexes from metalloprotease enzymes.

Thus, disclosed herein are CCCs and Fc-cytokine complexes with chimeric Fc-cytokine linkers or linker sequences that are protected from metalloprotease enzymes (see FIGS. 2 and 3). Such linkers were designed to not contain any predicted metalloprotease sites. Part of this effort involved using a protease site prediction tool called PROSPER [15].

One technical problem faced by the applicant is how to engineer a metalloprotease-resistant linker that promotes agonism or cell receptor binding. One technical solution discovered and developed by the applicant is to engineer the metalloprotease-resistant linkers such that the linker sequences comprise between 7 to 13 amino acid residues in length. This is supported by the literature which shows an inverse relationship between flexibility and agonism in the case of FcγRs [16]. The applicant predicts a similar relationship with Fc-cytokine chimeras due to a similar type of receptor clustering mechanism involved in both antibody [1] and cytokine agonism [2,3].

In some embodiments, the anti-CD3 and anti-CD28 T-cell activation reagent can comprise a plurality of self-assembling protein nanoparticles decorated with anti-CD3 antibodies and anti-CD28 antibodies. The self-assembling protein nanoparticles can comprise protein cage polypeptides assembled into three-dimensional structures.

In some embodiments, the three-dimensional structure of the protein cage polypeptide can be a tetrahedral pyramid. The protein cage polypeptides can also self-assemble into compact asymmetrical multimeric structures or cage-cage multimers (including dimers).

The three-dimensional structures (e.g., protein cage polypeptide formed into tetrahedral pyramids) can serve as scaffolds for the anti-CD3 antibodies and the anti-CD28 antibodies.

In some embodiments, the protein cage polypeptide can be comprised of a polypeptide comprising an amino acid sequence with at least about 70%, 75%, 80%, 85%, 90%, 95%, 99%, or 100% amino acid identity to the amino acid sequence set forth in any one of SEQ ID NOS: 8-10 (see Table 2).

The protein cage polypeptide can comprise a polypeptide of between about 400 and about 700 amino acid residues in length. In some embodiments, the protein cage polypeptide can comprise a polypeptide of about 450 amino acid residues to about 650 amino acid residues in length.

### Example 4: Chimeric Cytokine Complexes (CCCs) Enhance NK Cell Activation and Expansion

Another unexpected result stemming from the experiments disclosed herein is that CCCs enhance or markedly increase NK cell activation and expansion when compared to equivalent concentrations of soluble cytokines alone or Fc-cytokines alone.

Human peripheral blood CD56⁺ NK cells were thawed and media exchanged in serum free NK-cell expansion medium on day 0. The medium was supplemented with 10% human platelet lysate and one of the following: (1) standard soluble cytokines (e.g., IL-2s), (2) Fc-cytokine complexes (e.g., IL-2-Fcs), (3) CCCs (e.g., IL-2-Fcs), and (4) CCCs (e.g., IL-2-Fcs) with NK activators. For example, the NK activators can be a soluble NK activator for use in vitro.

NK cells were seeded at a concentration of 7×10⁵ cells/mL then placed in a 37° C. and 5% CO₂ incubator. On day 3, the cells were imaged under a light microscope.

Standard soluble cytokines were added to the medium at a concentration of 35 ng/mL and CCCs were added to the medium at concentrations equivalent (molar equivalent, m.e.) to those of the standard cytokines. The CCCs added were IL-2-Fc CCCs.

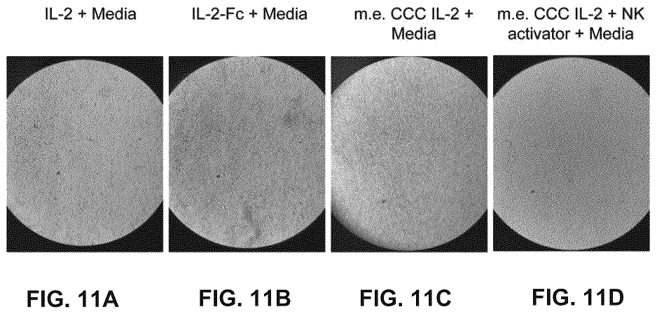

FIGS. 11A-1D illustrate NK cells imaged under a light microscope. As shown in FIGS. 11A-11D, donor peripheral blood CD56⁺ NK cells on media supplemented with CCCs (e.g., the IL-2-Fc CCCs and the IL-2-Fc CCCs with NK activators) proliferated much more than NK cells on media supplemented with standard soluble cytokines (e.g., IL-2s) alone or Fc-cytokines alone (e.g., Fc-IL-2s) after three days of coincubation.

A number of embodiments have been described. Nevertheless, it will be understood by one of ordinary skill in the art that various changes and modifications can be made to this disclosure without departing from the spirit and scope of the embodiments. Elements of systems, devices, apparatus, and methods shown with any embodiment are exemplary for the specific embodiment and can be used in combination or otherwise on other embodiments within this disclosure. For example, the steps of any methods depicted in the figures or described in this disclosure do not require the particular order or sequential order shown or described to achieve the desired results. In addition, other steps operations may be provided, or steps or operations may be eliminated or omitted from the described methods or processes to achieve the desired results. Moreover, any components or parts of any apparatus or systems described in this disclosure or depicted in the figures may be removed, eliminated, or omitted to achieve the desired results. In addition, certain components or parts of the systems, devices, or apparatus shown or described herein have been omitted for the sake of succinctness and clarity.

Accordingly, other embodiments are within the scope of the following claims and the specification and/or drawings may be regarded in an illustrative rather than a restrictive sense.

Each of the individual variations or embodiments described and illustrated herein has discrete components and features which may be readily separated from or combined with the features of any of the other variations or embodiments. Modifications may be made to adapt a particular situation, material, composition of matter, process, process act(s) or step(s) to the objective(s), spirit, or scope of the present invention.

Methods recited herein may be carried out in any order of the recited events that is logically possible, as well as the recited order of events. Moreover, additional steps or operations may be provided or steps or operations may be eliminated to achieve the desired result.

Furthermore, where a range of values is provided, every intervening value between the upper and lower limit of that range and any other stated or intervening value in that stated range is encompassed within the invention. Also, any optional feature of the inventive variations described may be set forth and claimed independently, or in combination with any one or more of the features described herein. For example, a description of a range from 1 to 5 should be considered to have disclosed subranges such as from 1 to 3, from 1 to 4, from 2 to 4, from 2 to 5, from 3 to 5, etc. as well as individual numbers within that range, for example 1.5, 2.5, etc. and any whole or partial increments therebetween.

All existing subject matter mentioned herein (e.g., publications, patents, patent applications, and journal articles) are incorporated by reference herein in their entireties except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise, all technical and scientific terms used herein have the same meaning as commonly understood by one of ordinary skill in the art to which this invention belongs.

Reference to the phrase “at least one of”, when such phrase modifies a plurality of items or components (or an enumerated list of items or components) means any combination of one or more of those items or components. For example, the phrase “at least one of A, B, and C” means: (i) A; (ii) B; (iii) C; (iv) A, B, and C; (v) A and B; (vi) B and C; or (vii) A and C.

In understanding the scope of the present disclosure, the term “comprising” and its derivatives, as used herein, are intended to be open-ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Also, the terms “part,” “section,” “portion,” “member” “element,” or “component” when used in the singular can have the dual meaning of a single part or a plurality of parts. As used herein, the following directional terms “forward, rearward, above, downward, vertical, horizontal, below, transverse, laterally, and vertically” as well as any other similar directional terms refer to those positions of a device or piece of equipment or those directions of the device or piece of equipment being translated or moved.

Finally, terms of degree such as “substantially”, “about” and “approximately” as used herein mean the specified value or the specified value and a reasonable amount of deviation from the specified value (e.g., a deviation of up to ±0.1%, ±1%, ±5%, or ±10%, as such variations are appropriate) such that the end result is not significantly or materially changed. For example, “about 1.0 cm” can be interpreted to mean “1.0 cm” or between “0.9 cm and 1.1 cm.” When terms of degree such as “about” or “approximately” are used to refer to numbers or values that are part of a range, the term can be used to modify both the minimum and maximum numbers or values.

The structures in the figures may be shown as distinct and communicating with only a few specific structures and not others. The structures may be merged with each other, may perform overlapping functions, and may communicate with other structures not shown to be connected in the figures. Accordingly, the specification and/or drawings may be regarded in an illustrative rather than a restrictive sense.

All cited references are hereby incorporated by reference in their entireties.

This disclosure is not intended to be limited to the scope of the particular forms set forth, but is intended to cover alternatives, modifications, and equivalents of the variations or embodiments described herein. Further, the scope of the disclosure fully encompasses other variations or embodiments that may become obvious to those skilled in the art in view of this disclosure.

### REFERENCES

[1] Mayes, P. A., Hance, K. W. & Hoos, A. The promise and challenges of immune agonist antibody development in cancer. Nat. Rev. Drug Discov. 17, 509-527 (2018).
[2] Spangler, J. B., Moraga, I., Mendoza, J. L. & Garcia, K. C. Insights into cytokine-receptor interactions from cytokine engineering. Annu. Rev. Immunol. 33, 139-167 (2015).
[3] Brooks, A. J. et al. Mechanism of activation of protein kinase JAK2 by the growth hormone receptor. Science 344, 1249783 (2014).
[4] O'Shea, J. J. & Plenge, R. JAK and STAT signaling molecules in immunoregulation and immune-mediated disease. Immunity 36, 542-550 (2012).
[5] Liao, W., Lin, J. X. & Leonard, W. J. Interleukin-2 at the crossroads of effector responses, tolerance, and immunotherapy. Immunity 38, 13-25 (2013).
[6] Levin, A. M. et al. Exploiting a natural conformational switch to engineer an interleukin-2 ‘superkine’. Nature 484, 529-533 (2012).
[7] Powell, M. S. & Hogarth, P. M. Fc receptors. Adv. Exp. Med. Biol. 640, 22-34 (2008).
[8] de Taeye, S. W. et al. FcgammaR Binding and ADCC Activity of Human IgG Allotypes. Front. Immunol. 11, 740 (2020).
[9] Shields, R. L. et al. High resolution mapping of the binding site on human IgG1 for Fc gamma RI, Fc gamma RII, Fc gamma RIII, and FcRn and design of IgG1 variants with improved binding to the Fc gamma R. J. Biol. Chem. 276, 6591-6604 (2001).
[10] Isoda, Y. et al. Importance of the Side Chain at Position 296 of Antibody Fc in Interactions with FcgammaRIIIa and Other Fcgamma Receptors. PLOS One 10, e0140120 (2015).
[11] Edsparr, K., Basse, P. H., Goldfarb, R. H. & Albertsson, P. Matrix metalloproteinases in cytotoxic lymphocytes impact on tumour infiltration and immunomodulation. Cancer Microenviron 4, 351-360 (2011).
[12] Benson, H. L. et al. Endogenous matrix metalloproteinases 2 and 9 regulate activation of CD4+ and CD8+ T cells. Am J Respir Cell Mol Biol 44, 700-708 (2011).
[13] Johnatty, R. N. et al. Cytokine and chemokine regulation of proMMP-9 and TIMP-1 production by human peripheral blood lymphocytes. J. Immunol. 158, 2327-2333 (1997).
[14] de Almeida, L. G. N. et al. Matrix Metalloproteinases: From Molecular Mechanisms to Physiology, Pathophysiology, and Pharmacology. Pharmacol. Rev. 74, 712-768 (2022).
[15] Song, J. et al. PROSPER: an integrated feature-based tool for predicting protease substrate cleavage sites. PLOS One 7, e50300 (2012).
[16] Liu, X. et al. Human immunoglobulin G hinge regulates agonistic anti-CD40 immunostimulatory and antitumour activities through biophysical flexibility. Nature Communications 10, 4206 (2019).

We claim:

1. A method of activating and expanding immune cells, comprising:
adding a chimeric cytokine complex to a population of immune cells, wherein the chimeric cytokine complex comprises:
a protein cage polypeptide, wherein the protein cage polypeptide comprises polypeptides that self-assemble into a tetrahedral pyramid structure;
a plurality of engineered Fc antibody domains non-covalently bound to the protein cage polypeptide; and
one or more cytokines covalently linked to each of the plurality of engineered Fc antibody domains.

2. The method of claim 1, wherein the population of immune cells are activated and expanded in vitro.

3. The method of claim 1, wherein the population of immune cells are human donor peripheral blood immune cells.

4. The method of claim 1, wherein the population of immune cells are live T cells.

5. The method of claim 1, wherein the population of immune cells are natural killer (NK) cells.

6. The method of claim 4, further comprising activating the population of T cells with an anti-CD3 and anti-CD28 T-cell activation reagent prior to adding the chimeric cytokine complex.

7. The method of claim 1, wherein the cytokines are interleukins.

8. The method of claim 7, wherein the interleukins are at least one of interleukin-2s (IL-2s), IL-7s, and IL-15s.

9. The method of claim 1, wherein at least one of the engineered Fc antibody domains is C-terminally linked to an N-terminus of at least one of the cytokines.

10. The method of claim 1, wherein at least one of the engineered Fc antibody domains is N-terminally linked to a C-terminus of at least one of the cytokines.

11. The method of claim 1, wherein the protein cage polypeptide is comprised of a polypeptide comprising an amino acid sequence with at least about 70%, 75%, 80%, 85%, 90%, 95%, 99%, or 100% amino acid identity to the amino acid sequence set forth in any one of SEQ ID NOS: 8-15.

12. The method of claim 1, wherein the protein cage polypeptide is comprised of a polypeptide comprising a binding site for one of the engineered Fc antibody domains, and wherein the binding site comprises the amino acid sequence RWGSGADCAWHLGELVWCTAGSGWE (SEQ ID NO:16).

13. The method of claim 1, wherein the protein cage polypeptide is comprised of a polypeptide comprising a binding site for one of the engineered Fc antibody domains, and wherein the binding site comprises the amino acid sequence GGRWGADCAWHLGELVWCTAGWEGG (SEQ ID NO:17).

14. The method of claim 1, wherein the protein cage polypeptide is comprised of a polypeptide comprising a binding site for one of the engineered Fc antibody domains, and wherein the binding site comprises the amino acid sequence GADCAWHLGELVWCTAG (SEQ ID NO:18).

15. The method of claim 1, wherein the engineered Fc antibody domains are engineered human Fc antibody domains.

16. The method of claim 1, wherein the engineered Fc antibody domains are engineered rabbit Fc antibody domains.

17. A method of activating and expanding immune cells, comprising:
adding a chimeric cytokine complex to a population of immune cells, wherein the chimeric cytokine complex comprises:
a protein cage polypeptide;
a plurality of engineered Fc antibody domains non-covalently bound to the protein cage polypeptide, wherein the plurality of engineered Fc antibody domains comprises between six and twelve engineered Fc antibody domains non-covalently bound to the protein cage polypeptide; and
one or more cytokines covalently linked to each of the plurality of engineered Fc antibody domains.

18. The method of claim 17, wherein between 12 and 24 cytokines are linked to the plurality of engineered Fc antibody domains in total.

19. A method of activating and expanding immune cells, comprising:
adding a chimeric cytokine complex to a population of immune cells, wherein the chimeric cytokine complex comprises:
a protein cage polypeptide;
a plurality of engineered Fc antibody domains non-covalently bound to the protein cage polypeptide; and
one or more cytokines covalently linked to each of the plurality of engineered Fc antibody domains, wherein each of the one or more cytokines is covalently linked to one of the engineered Fc antibody domains via an engineered metalloprotease-resistant linker sequence.

20. The method of claim 19, wherein the engineered metalloprotease-resistant linker sequence is between 7 to 13 amino acid residues in length.


SEQUENCE LISTING (32 sequences)

SEQ ID NO: 1 - protein, 231 aa, synthetic construct
PKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 383 aa, synthetic construct
MGWSCIILFLVATATGVHSPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 3 - protein, 383 aa, synthetic construct
MGWSCIILFLVATATGVHSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 402 aa, synthetic construct
MGWSCIILFLVATATGVHSPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 5 - protein, 402 aa, synthetic construct
MGWSCIILFLVATATGVHSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 364 aa, synthetic construct
MGWSCIILFLVATATGVHSPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 7 - protein, 364 aa, synthetic construct
MGWSCIILFLVATATGVHSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 478 aa, synthetic construct
MPFITVGQENSTSIDLYYEDHGTGTPVVLIHGFPLSGHSWERQSAALLDAGYRVITYDRRGFGQSSQPTTGYDYDTFAADLNTVLETLDLQDAVLVGFSMGTGEVARYVSSYGTARIAAVAFLASLEPFLLKTDDNPDGAAPQEFFDGIVAAVKADRYAFYTGFFNDFYNLDENLGTRISEEAVRNSWNTAASGGFFAAAAAPTTWYTDFRADIPRIDVPALILHGTGDRTLPIENTARVFHKALPSAEYVEVEGAPHGLLWTHAEEVNTALLAFLAKAQEAQKQKLLTEVETYVLSIIPSGPLKAEIAQRLEDVFAGRWGSGADCAWHLGELVWCTAGSGWEDLEVLMEWLKTRPILSPLTKGILGFVFTLTVPSERGLQRRRFVQNALNGNGDPNNMDKAVKLYRKLKREITFHGAKEISLSYSAGALASCMGLIYNRMGAVTTEVAFGLVCATCEQIADSQHRSHRQLEHHHHHH

SEQ ID NO: 9 - protein, 478 aa, synthetic construct
MPFITVGQENSTSIDLYYEDHGTGTPVVLIHGFPLSGHSWERQSAALLDAGYRVITYDRRGFGQSSQPTTGYDYDTFAADLNTVLETLDLQDAVLVGFSMGTGEVARYVSSYGTARIAAVAFLASLEPFLLKTDDNPDGAAPQEFFDGIVAAVKADRYAFYTGFFNDFYNLDENLGTRISEEAVRNSWNTAASGGFFAAAAAPTTWYTDFRADIPRIDVPALILHGTGDRTLPIENTARVFHKALPSAEYVEVEGAPHGLLWTHAEEVNTALLAFLAKAQEAQKQKLLTEVETYVLSIIPSGPLKAEIAQRLEDVFAGGGRWGADCAWHLGELVWCTAGWEGGDLEVLMEWLKTRPILSPLTKGILGFVFTLTVPSERGLQRRRFVQNALNGNGDPNNMDKAVKLYRKLKREITFHGAKEISLSYSAGALASCMGLIYNRMGAVTTEVAFGLVCATCEQIADSQHRSHRQLEHHHHHH

SEQ ID NO: 10 - protein, 470 aa, synthetic construct
MPFITVGQENSTSIDLYYEDHGTGTPVVLIHGFPLSGHSWERQSAALLDAGYRVITYDRRGFGQSSQPTTGYDYDTFAADLNTVLETLDLQDAVLVGFSMGTGEVARYVSSYGTARIAAVAFLASLEPFLLKTDDNPDGAAPQEFFDGIVAAVKADRYAFYTGFFNDFYNLDENLGTRISEEAVRNSWNTAASGGFFAAAAAPTTWYTDFRADIPRIDVPALILHGTGDRTLPIENTARVFHKALPSAEYVEVEGAPHGLLWTHAEEVNTALLAFLAKAQEAQKQKLLTEVETYVLSIIPSGPLKAEIAQRLEDVFAGGADCAWHLGELVWCTAGDLEVLMEWLKTRPILSPLTKGILGFVFTLTVPSERGLQRRRFVQNALNGNGDPNNMDKAVKLYRKLKREITFHGAKEISLSYSAGALASCMGLIYNRMGAVTTEVAFGLVCATCEQIADSQHRSHRQLEHHHHHH

SEQ ID NO: 11 - protein, 478 aa, synthetic construct
MPFITVGQENSTSIDLYYEDHGTGTPVVLIHGFPLSGHSWERQSAALLDAGYRVITYDRRGFGQSSQPTTGYDYDTFAADLNTVLETLDLQDAVLVGFSMGTGEVARYVSSYGTARIAAVAFLASLEPFLLKTDDNPDGAAPQEFFDGIVAAVKADRYAFYTGFFNDFYNLDENLGTRISEEAVRNSWNTAASGGFFAAAAAPTTWYTDFRADIPRIDVPALILHGTGDRTLPIENTARVFHKALPSAEYVEVEGAPHGLLWTHAEEVNTALLAFLAKAQEAQKQKLLTEVETYVLSIIPSGPLKAEIAQRLEDVFAGRWGSGCDCAWHLGELVWCTCGSGWEDLEVLMEWLKTRPILSPLTKGILGFVFTLTVPSERGLQRRRFVQNALNGNGDPNNMDKAVKLYRKLKREITFHGAKEISLSYSAGALASCMGLIYNRMGAVTTEVAFGLVCATCEQIADSQHRSHRQLEHHHHHH

SEQ ID NO: 12 - protein, 478 aa, synthetic construct
MPFITVGQENSTSIDLYYEDHGTGTPVVLIHGFPLSGHSWERQSAALLDAGYRVITYDRRGFGQSSQPTTGYDYDTFAADLNTVLETLDLQDAVLVGFSMGTGEVARYVSSYGTARIAAVAFLASLEPFLLKTDDNPDGAAPQEFFDGIVAAVKADRYAFYTGFFNDFYNLDENLGTRISEEAVRNSWNTAASGGFFAAAAAPTTWYTDFRADIPRIDVPALILHGTGDRTLPIENTARVFHKALPSAEYVEVEGAPHGLLWTHAEEVNTALLAFLAKAQEAQKQKLLTEVETAVLSIIPSGPLKAEIAQRLEDVFAGRWGSGADCAWHLGELVWCTAGSGWEDLEVLMEWLKTRPILSPLTKGILGFVFTLTVPSERGLQRRRFVQNALNGNGDPNNMDKAVKLYRKLKREITFHGAKEISLSYSAGALASCMGLIYNRMGAVTTEVAFGLVCATCEQIADSQHRSHRQLEHHHHHH

SEQ ID NO: 13 - protein, 478 aa, synthetic construct
MPFITVGQENSTSIDLYYEDHGTGTPVVLIHGFPLSGHSWERQSAALLDAGYRVITYDRRGFGQSSQPTTGYDYDTFAADLNTVLETLDLQDAVLVGFSMGTGEVARYVSSYGTARIAAVAFLASLEPFLLKTDDNPDGAAPQEFFDGIVAAVKADRYAFYTGFFNDFYNLDENLGTRISEEAVRNSWNTAASGGFFAAAAAPTTWYTDFRADIPRIDVPALILHGTGDRTLPIENTARVFHKALPSAEYVEVEGAPHGLLWTHAEEVNTALLAFLAKAQEAQKQKLLTEVETAVLSIIPSGPLKAEIAQRLEDVFAGGGRWGADCAWHLGELVWCTAGWEGGDLEVLMEWLKTRPILSPLTKGILGFVFTLTVPSERGLQRRRFVQNALNGNGDPNNMDKAVKLYRKLKREITFHGAKEISLSYSAGALASCMGLIYNRMGAVTTEVAFGLVCATCEQIADSQHRSHRQLEHHHHHH

SEQ ID NO: 14 - protein, 470 aa, synthetic construct
MPFITVGQENSTSIDLYYEDHGTGTPVVLIHGFPLSGHSWERQSAALLDAGYRVITYDRRGFGQSSQPTTGYDYDTFAADLNTVLETLDLQDAVLVGFSMGTGEVARYVSSYGTARIAAVAFLASLEPFLLKTDDNPDGAAPQEFFDGIVAAVKADRYAFYTGFFNDFYNLDENLGTRISEEAVRNSWNTAASGGFFAAAAAPTTWYTDFRADIPRIDVPALILHGTGDRTLPIENTARVFHKALPSAEYVEVEGAPHGLLWTHAEEVNTALLAFLAKAQEAQKQKLLTEVETAVLSIIPSGPLKAEIAQRLEDVFAGGADCAWHLGELVWCTAGDLEVLMEWLKTRPILSPLTKGILGFVFTLTVPSERGLQRRRFVQNALNGNGDPNNMDKAVKLYRKLKREITFHGAKEISLSYSAGALASCMGLIYNRMGAVTTEVAFGLVCATCEQIADSQHRSHRQLEHHHHHH

SEQ ID NO: 15 - protein, 478 aa, synthetic construct
MPFITVGQENSTSIDLYYEDHGTGTPVVLIHGFPLSGHSWERQSAALLDAGYRVITYDRRGFGQSSQPTTGYDYDTFAADLNTVLETLDLQDAVLVGFSMGTGEVARYVSSYGTARIAAVAFLASLEPFLLKTDDNPDGAAPQEFFDGIVAAVKADRYAFYTGFFNDFYNLDENLGTRISEEAVRNSWNTAASGGFFAAAAAPTTWYTDFRADIPRIDVPALILHGTGDRTLPIENTARVFHKALPSAEYVEVEGAPHGLLWTHAEEVNTALLAFLAKAQEAQKQKLLTEVETAVLSIIPSGPLKAEIAQRLEDVFAGRWGSGCDCAWHLGELVWCTCGSGWEDLEVLMEWLKTRPILSPLTKGILGFVFTLTVPSERGLQRRRFVQNALNGNGDPNNMDKAVKLYRKLKREITFHGAKEISLSYSAGALASCMGLIYNRMGAVTTEVAFGLVCATCEQIADSQHRSHRQLEHHHHHH

SEQ ID NO: 16 - protein, 25 aa, synthetic construct
RWGSGADCAWHLGELVWCTAGSGWE

SEQ ID NO: 17 - protein, 25 aa, synthetic construct
GGRWGADCAWHLGELVWCTAGWEGG

SEQ ID NO: 18 - protein, 17 aa, synthetic construct
GADCAWHLGELVWCTAG

SEQ ID NO: 19 - protein, 25 aa, synthetic construct
RWGSGCDCAWHLGELVWCTCGSGWE

SEQ ID NO: 20 - protein, 10 aa, synthetic construct
SLSPGKAPTS

SEQ ID NO: 21 - protein, 13 aa, synthetic construct
SLSPGKDCDIEGK

SEQ ID NO: 22 - protein, 7 aa, synthetic construct
SLSPGKN

SEQ ID NO: 23 - protein, 10 aa, synthetic construct
TPKSCDKTHT

SEQ ID NO: 24 - protein, 10 aa, synthetic construct
HPKSCDKTHT

SEQ ID NO: 25 - protein, 11 aa, synthetic construct
TSPKSCDKTHT

SEQ ID NO: 26 - protein, 19 aa, synthetic construct
MGWSCIILFLVATATGVHS

SEQ ID NO: 27 - protein, 70 aa, synthetic construct
QQGNVFSCSVMHEALHNHYTQKSLSLSPGKAPTSSSTKKTQLQLEHLLLDLQMILNGINNYKNPKLTRML

SEQ ID NO: 28 - protein, 54 aa, synthetic construct
WITFCQSIISTLTPKSCDKTHTCPPCPAPELLGGPSVFLFPPKPKDTLMISRTP

SEQ ID NO: 29 - protein, 70 aa, synthetic construct
SRWQQGNVFSCSVMHEALHNHYTQKSLSLSPGKDCDIEGKDGKQYESVLMVSIDQLLDSMKEIGSNCLNN

SEQ ID NO: 30 - protein, 74 aa, synthetic construct
DLCFLKRLLQEIKTCWNKILMGTKEHPKSCDKTHTCPPCPAPELLGGPSVFLFPPKPKDTLMISRTPEVTCVVV

SEQ ID NO: 31 - protein, 68 aa, synthetic construct
WQQGNVFSCSVMHEALHNHYTQKSLSLSPGKNWVNVISDLKKIEDLIQSMHIDATLYTESDVHPSCKV

SEQ ID NO: 32 - protein, 43 aa, synthetic construct
FVHIVQMFINTSPKSCDKTHTCPPCPAPELLGGPSVFLFPPKP